(19) United States
(12) Patent Application Publication
(10) Pub. No.: US 2015/0342959 A1
(43) Pub. Date: Dec. 3, 2015

(54) METHODS AND COMPOSITIONS FOR SUSTAINED NORIBOGAINE TREATMENT

(71) Applicant: DemeRx, Inc., Fort Lauderdale, FL (US)

(72) Inventor: Lawrence Friedhoff, River Vale, NJ (US)

(73) Assignee: DemeRx, Inc., Fort Lauderdale, FL (US)

(21) Appl. No.: 14/624,399

(22) Filed: Feb. 17, 2015

Related U.S. Application Data
(60) Provisional application No. 62/007,346, filed on Jun. 3, 2014.

(51) Int. Cl. A61K 31/55

(52) U.S. Cl. CPC A61K 31/55

(57) ABSTRACT

This invention is directed to a method of treating opioid or opioid-like drug addiction, including acute and post-acute withdrawal symptoms, comprising treating an addicted patient with noribogaine at a dosage that provides an average serum concentration of 50 ng/mL to 180 ng/mL under conditions where the QT interval prolongation does not exceed about 50 milliseconds.

### CROSS-REFERENCE TO RELATED APPLICATIONS

This application claims benefit from U.S. Provisional Application No. 62/007,346, filed Jun. 3, 2014, which is hereby incorporated by reference in its entirety.

### FIELD OF THE INVENTION

This invention is directed to a method for treating a patient while maintaining an acceptable QT interval prolongation in the patient, comprising administering to the patient an initial unit dose of noribogaine, noribogaine derivative, or pharmaceutically acceptable salt or solvate thereof.

### BACKGROUND OF THE INVENTION

Noribogaine is sometimes referred to as 12-hydroxyibogaine. U.S. Pat. No. 2,813,873 claims noribogaine albeit as “12-O-demethylibogaine” while providing an incorrect structural formula for ibogaine. The structure of noribogaine has now been thoroughly evaluated and is found to combine the features of tryptamine, tetrahydrohavaine and indolazepines. Noribogaine can be depicted by the following formula:

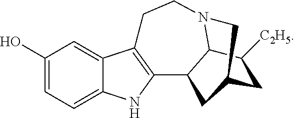

Noribogaine and its pharmaceutically acceptable salts have recently received significant attention as a non-addictive alkaloid useful in treating drug dependency (U.S. Pat. No. 6,348,456) and as a potent analgesic (U.S. Pat. No. 7,220,737). Such treatment generally requires administration of high doses of noribogaine, typically 0.1 mg to 100 mg per kg body weight.

Noribogaine is a metabolite of ibogaine found in human, dog, rat and monkey. While the prior art suggests that ibogaine at higher doses is useful as a treatment for addiction, use of ibogaine is associated with hallucinations and other negative side effects. In the United States, ibogaine is classified as a Schedule I controlled substance. Noribogaine has been suggested to have a greater and longer lasting activity in humans than ibogaine for reducing craving for addictive substances and treating chemical dependency. U.S. Pat. No. 6,348,456, incorporated by reference herein in its entirety, discloses highly purified noribogaine and teaches that it should be provided at dosages from about 0.01 to about 100 mg per kg body weight per day to treat addiction, although no human data was provided showing an effective dose to treat drug addiction.

The therapeutic dosing of noribogaine for long-term treatment in humans has not previously been addressed, especially as it relates to dosing protocols that are effective, as well as safe. Indeed, prior to the instant invention, it was uncertain as to whether noribogaine could be administered at a dose which was therapeutic while at the same time safe for patients.

### SUMMARY

While noribogaine has been disclosed for treatment of several conditions, including drug addiction, its use in humans is complicated by the fact that the ranges in the prior art are exceptionally broad (0.01 to 1000 mg/kg body weight). Human clinical studies demonstrate that the lower dosing of noribogaine has minimal impact on withdrawal symptoms in addicted patients. Doses that are too high are similarly sub-effective. Thus, the previously disclosed broad range has now been found to be insufficient for human therapy at the lower end of this range.

Moreover, the use of noribogaine imparts a dose dependent prolongation of the treated patient's QT interval, rendering higher dosing of noribogaine unacceptable. A prolonged QT interval is a marker of potential Torsades de Pointes, a serious arrhythmia that can result in death.

The current invention is predicated on the surprising discovery that treatment with a narrow dosage range of noribogaine, noribogaine derivative, or pharmaceutically acceptable salt or solvate thereof provides a therapeutic blood serum concentration in treated patients.

According to the present invention, a method for treating a condition in a patient which is treatable with noribogaine, noribogaine derivative, or pharmaceutically acceptable salt or solvate thereof, while maintaining an acceptable QT interval prolongation in said patient, the method comprising:

a) administering to the patient an initial unit dose of noribogaine, noribogaine derivative, or pharmaceutically acceptable salt or solvate thereof, wherein said unit dose provides a therapeutic average serum concentration of 50 ng/mL to 180 ng/mL which serum concentration imparts minimal QT interval prolongation; and

b) maintaining said serum concentration by periodically administering at least one additional dose of noribogaine, noribogaine derivative, or pharmaceutically acceptable salt or solvate thereof, such that the at least one additional dose maintains the average serum concentration of 50 ng/mL to 180 ng/mL during treatment

wherein said additional dose or doses are continued as necessary to treat said condition.

In one embodiment, the therapeutic blood serum concentration is between 50 ng/mL and 180 ng/mL. In a preferred embodiment, the therapeutic serum concentration is 80 ng/mL to 100 ng/mL. In one embodiment, the dose range provides both therapeutic results and an acceptable QT interval prolongation of less than 50 milliseconds in a patient. In one embodiment, an initial unit dose of noribogaine, noribogaine derivative, or pharmaceutically acceptable salt or solvate thereof is administered. In one embodiment, the initial unit dose provides the therapeutic blood serum concentration with minimal QT interval prolongation. In one embodiment, the QT interval is not prolonged by more than 20 ms.

In some embodiments, the patient is administered an initial unit dose of noribogaine, noribogaine derivative, or pharmaceutically acceptable salt or solvate thereof, followed by one or more additional doses. In one embodiment, the initial unit dose is from 50 mg to 120 mg. In one embodiment, the one or more additional doses are lower than the initial dose. In one embodiment, the one or more additional doses are from 5 mg to 50 mg. In one embodiment, such a dosing regimen provides a therapeutic average serum concentration of noribogaine of 50 ng/mL to 180 ng/mL. In one embodiment, the one or more additional doses maintain a therapeutic average serum concentration of 50 ng/mL to 180 ng/mL over a period of time. In one embodiment, the one or more additional doses are administered periodically. In one embodiment, the at least one additional dose is administered between about 6 hours and about 24 hours after administration of the initial unit dose. In one embodiment, the at least one additional dose is administered between about 6 hours and about 24 hours after administration of the previous dose. In one embodiment, one or more doses are administered as a controlled release formulation.

In a preferred embodiment, the narrow therapeutic doses of noribogaine, noribogaine derivative, or pharmaceutically acceptable salt or solvate described above unexpectedly do not prolong the QT interval to unacceptable levels in human patients. In some embodiments, the patient will be pre-screened to evaluate tolerance for prolongation of QT interval, e.g., to determine whether the patient has any pre-existing cardiac conditions which would disqualify them from treatment with noribogaine. In some embodiments, the patient undergoes cardiac monitoring during a portion of the treatment. In a preferred embodiment, cardiac monitoring is not required. In one embodiment, the patient is tested to determine QT interval before treatment with noribogaine, and if clinician determines that the QT prolongation would be unacceptable risk, noribogaine therapy will be contraindicated.

### BRIEF DESCRIPTION OF THE FIGURES

FIG. 1 represents mean noribogaine concentration-time profiles in healthy patients after single oral dosing with 3, 10, 30 or 60 mg doses. Inset: Individual concentration-time profiles from 0-12 h after a 10 mg dose.

FIG. 2 represents mean plasma noribogaine glucuronide concentration-time profiles in healthy patients after single oral 30 or 60 mg doses.

FIG. 3 illustrates the mean noribogaine concentration-time profile in opioid-addicted patients after a single oral 60 mg (diamonds), 120 mg (squares), or 180 mg (triangles) dose of noribogaine.

FIG. 4 illustrates hours to resumption of opioid substitution treatment (OST) for each patient given placebo (circles), or a single oral dose of noribogaine (60 mg, squares; 120 mg, triangles; 180 mg, inverted triangles). Center horizontal line represents mean. Error bars represent standard deviation.

FIG. 5 illustrates results of noribogaine treatment on final COWS scores before resumption of OST. Boxes include values representing 25%-75% quartiles. Diamonds represent the median, crossbars represent mean. Whiskers represent values within one standard deviation of mid-quartiles. No outliers were present.

FIG. 6A illustrates of the mean change in total COWS scores over the first 6 hours following dosing of noribogaine (60 mg, squares; 120 mg, triangles; 180 mg, diamonds) or placebo (circles). Data is given relative to baseline COWS score.

FIG. 6B illustrates the mean area under the curve (AUC) over the initial 6 hour period after administration of noribogaine or placebo, based on the COWS score data given in FIG. 6A. A negative change in score indicates that withdrawal symptoms subsided over the period.

FIG. 7A illustrates of the mean change in total OOWS scores over the first 6 hours following dosing of noribogaine (60 mg, squares; 120 mg, triangles; 180 mg, diamonds) or placebo (circles). Data is given relative to baseline OOWS score.

FIG. 7B illustrates the mean area under the curve (AUC) over the initial 6 hour period after administration of noribogaine or placebo, based on the OOWS score data given in FIG. 7A. A negative change in score indicates that withdrawal symptoms subsided over the period.

FIG. 8A illustrates of the mean change in total SOWS scores over the first 6 hours following dosing of noribogaine (60 mg, squares; 120 mg, triangles; 180 mg, diamonds) or placebo (circles). Data is given relative to baseline SOWS score.

FIG. 8B illustrates the mean area under the curve (AUC) over the initial 6 hour period after administration of noribogaine or placebo, based on the SOWS score data given in FIG. 8A. A negative change in score indicates that withdrawal symptoms subsided over the period.

FIG. 9A illustrates the average change in QT interval (ΔQTcl) for each cohort (60 mg, squares; 120 mg, triangles; 180 mg, diamonds) or placebo (circles) over the first 24 hours post administration.

FIG. 9B illustrates the correlation between serum noribogaine concentration and ΔQTcl for each patient over time. The equation of the line is given.

FIG. 10A illustrates the expected noribogaine serum concentration over time after noribogaine treatment using a dosing protocol with an initial dose of 80 mg and subsequent doses of 15 mg to 25 mg at 6 hour or 12 hour intervals.

FIG. 10B illustrates the expected noribogaine serum concentration over time after noribogaine treatment using a dosing protocol with an initial dose of 80 mg, administered as subdoses every 2 hours, and subsequent doses of 15 mg to 25 mg at 6 hour or 12 hour intervals.

### DETAILED DESCRIPTION

It is to be understood that this invention is not limited to particular embodiments described, as such may, of course, vary. It is also to be understood that the terminology used herein is for the purpose of describing particular embodiments only, and is not intended to be limiting, since the scope of this invention will be limited only by the appended claims.

The detailed description of the invention is divided into various sections only for the reader's convenience and disclosure found in any section may be combined with that in another section. Unless defined otherwise, all technical and scientific terms used herein have the same meaning as commonly understood by one of ordinary skill in the art to which this invention belongs.

It must be noted that as used herein and in the appended claims, the singular forms “a”, “an”, and “the” include plural referents unless the context clearly dictates otherwise. Thus, for example, reference to “a compound” includes a plurality of compounds.

### I. Definitions

Unless defined otherwise, all technical and scientific terms used herein have the same meaning as commonly understood by one of ordinary skill in the art to which this invention belongs. As used herein the following terms have the following meanings.

The term “about” when used before a numerical designation, e.g., temperature, time, amount, concentration, and such other, including a range, indicates approximations which may vary by (+) or (−) 20%, 10%, 5% or 1%, or any subrange or subvalue there between. Preferably, the term “about” when used with regard to a dose amount means that the dose may vary by +/−20%. For example, “about 2 mg/kg noribogaine” indicates that a patient may be administered a dose of noribogaine between 1.6 mg/kg and 2.4 mg/kg. In another example, about 120 mg per unit dose of noribogaine indicates that the unit dose may range from 96 mg to 144 mg.

“Administration” refers to introducing an agent, such as noribogaine, into a patient. Typically, an effective amount is administered, which amount can be determined by the treating physician or the like. Any route of administration, such as oral, topical, subcutaneous, peritoneal, intra-arterial, inhalation, vaginal, rectal, nasal, introduction into the cerebrospinal fluid, or instillation into body compartments can be used. The agent, such as noribogaine, may be administered by direct blood stream delivery, e.g. sublingual, buccal, intranasal, or intrapulmonary administration.

The related terms and phrases “administering” and “administration of”, when used in connection with a compound or pharmaceutical composition (and grammatical equivalents) refer both to direct administration, which may be administration to a patient by a medical professional or by self-administration by the patient, and/or to indirect administration, which may be the act of prescribing a drug. For example, a physician who instructs a patient to self-administer a drug and/or provides a patient with a prescription for a drug is administering the drug to the patient.

“Periodic administration” or “periodically administering” refers to multiple treatments that occur on a daily, weekly, or monthly basis. Periodic administration may also refer to administration of noribogaine, noribogaine derivative, or salt or solvate thereof one, two, three, or more times per day. Administration may be via transdermal patch, gum, lozenge, sublingual tablet, intranasal, intrapulmonary, oral administration, or other administration.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

As used herein, the term “alkyl” refers to monovalent saturated aliphatic hydrocarbyl groups having from 1 to 12 carbon atoms, 1 to 10 carbon atoms, preferably 1 to 6 carbon atoms, and more preferably 1 to 3 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—). The term “Cₓ alkyl” refers to an alkyl group having x carbon atoms, wherein x is an integer, for example, C₃ refers to an alkyl group having 3 carbon atoms.

“Alkenyl” refers to straight or branched hydrocarbyl groups having from 2 to 6 carbon atoms and preferably 2 to 4 carbon atoms and having at least 1 and preferably from 1 to 2 sites of vinyl (>C═C<) unsaturation. Such groups are exemplified, for example, by vinyl, allyl, and but-3-en-1-yl. Included within this term are the cis and trans isomers or mixtures of these isomers.

“Alkynyl” refers to straight or branched monovalent hydrocarbyl groups having from 2 to 6 carbon atoms and preferably 2 to 3 carbon atoms and having at least 1 and preferably from 1 to 2 sites of acetylenic (—C≡C—) unsaturation. Examples of such alkynyl groups include acetylenyl (—C≡CH), and propargyl (—CH₂C≡CH).

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to a vinyl (unsaturated) carbon atom.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to an acetylenic carbon atom.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Acyl includes the “acetyl” group CH₃C(O)—.

“Acylamino” refers to the groups —NR³⁸C(O)alkyl, —NR³⁸C(O)substituted alkyl, —NR³⁸C(O)cycloalkyl, —NR³⁸C(O)substituted cycloalkyl, —NR³⁸C(O)cycloalkenyl, —NR³⁸C(O)substituted cycloalkenyl, —NR³⁸C(O)alkenyl, —NR³⁸C(O)substituted alkenyl, —NR³⁸C(O)alkynyl, —NR³⁸C(O)substituted alkynyl, —NR³⁸C(O)aryl, —NR³⁸C(O)substituted aryl, —NR³⁸C(O)heteroaryl, —NR³⁸C(O)substituted heteroaryl, —NR³⁸C(O)heterocyclic, and —NR³⁸C(O)substituted heterocyclic wherein R³⁸ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group —NH₂.

“Substituted amino” refers to the group —NR³⁹R⁴⁰ where R³⁹ and R⁴⁰ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cycloalkyl, —SO₂-cycloalkenyl, —SO₂-substituted cycloalkenyl, —SO₂-aryl, —SO₂-substituted aryl, —SO₂-heteroaryl, —SO₂-substituted heteroaryl, —SO₂-heterocyclic, and —SO₂-substituted heterocyclic and wherein R³⁹ and R⁴⁰ are optionally joined, together with the nitrogen bound thereto to form a heterocyclic or substituted heterocyclic group, provided that R³⁹ and R⁴⁰ are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. When R³⁹ is hydrogen and R⁴⁰ is alkyl, the substituted amino group is sometimes referred to herein as alkylamino. When R³⁹ and R⁴⁰ are alkyl, the substituted amino group is sometimes referred to herein as dialkylamino. When referring to a monosubstituted amino, it is meant that either R³⁹ or R⁴⁰ is hydrogen but not both. When referring to a disubstituted amino, it is meant that neither R³⁹ nor R⁴⁰ are hydrogen.

“Aminocarbonyl” refers to the group —C(O)NR⁴¹R⁴² where R⁴¹ and R⁴² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁴¹ and R⁴² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonyl” refers to the group —C(S)NR⁴¹R⁴² where R⁴¹ and R⁴² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁴¹ and R⁴² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR³⁸C(O)NR⁴¹R⁴² where R³⁸ is hydrogen or alkyl and R⁴¹ and R⁴² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁴¹ and R⁴² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonylamino” refers to the group —NR³⁸C(S)NR⁴¹R⁴² where R³⁸ is hydrogen or alkyl and R⁴¹ and R⁴² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁴¹ and R⁴² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyloxy” refers to the group —O—C(O)NR⁴¹R⁴² where R⁴¹ and R⁴² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁴¹ and R⁴² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyl” refers to the group —SO₂NR⁴¹R⁴² where R^{41′} and R⁴² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁴¹ and R⁴² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyloxy” refers to the group —O—SO₂NR⁴¹R⁴² where R⁴¹ and R⁴² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁴¹ and R⁴² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonylamino” refers to the group —NR³⁸—SO₂NR⁴¹R⁴² where R³⁸ is hydrogen or alkyl and R⁴¹ and R⁴² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁴¹ and R⁴² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amidino” refers to the group —C(═NR⁴³)NR⁴¹R⁴² where R⁴¹, R⁴², and R⁴³ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁴¹ and R⁴² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

“Substituted aryl” refers to aryl groups which are substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Carbonyl” refers to the divalent group —C(O)— which is equivalent to —C(═O)—.

“Carboxy” or “carboxyl” refers to —COOH or salts thereof.

“Carboxyl ester” or “carboxy ester” refers to the
groups —C(O)O-alkyl, —C(O)O-substituted alkyl, —C(O)O-alkenyl, —C(O)O-substituted
alkenyl, —C(O)O-alkynyl, —C(O)O-substituted alkynyl, —C(O)O-aryl, —C(O)O-substituted
aryl, —C(O)O-cycloalkyl, —C(O)O-substituted
cycloalkyl, —C(O)O-cycloalkenyl, —C(O)O-substituted
cycloalkenyl, —C(O)O-heteroaryl, —C(O)O-substituted heteroaryl, —C(O)O-heterocyclic,
   and —C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)amino” refers to the
group —NR³⁸—C(O)O-alkyl, —NR³⁸—C(O)O-substituted
alkyl, —NR³⁸—C(O)O-alkenyl, —NR³⁸—C(O)O-substituted
alkenyl, —NR³⁸—C(O)O-alkynyl, —NR³⁸—C(O)O-substituted
alkynyl, —NR³⁸—C(O)O-aryl, —NR³⁸—C(O)O-substituted
aryl, —NR³⁸—C(O)O-cycloalkyl, —NR³⁸—C(O)O-substituted
cycloalkyl, —NR³⁸—C(O)O-cycloalkenyl, —NR³⁸—C(O)O-substituted
cycloalkenyl, —NR³⁸—C(O)O-heteroaryl, —NR³⁸—C(O)O-substituted
   heteroaryl, —NR³⁸—C(O)O-heterocyclic, and —NR³⁸—C(O)O-substituted heterocyclic wherein R³⁸ is alkyl or hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” refers to the group —O—C(O)O-alkyl,
substituted —O—C(O)O-alkyl, —O—C(O)O-alkenyl, —O—C(O)O-substituted
alkenyl, —O—C(O)O-alkynyl, —O—C(O)O-substituted
alkynyl, —O—C(O)O-aryl, —O—C(O)O-substituted
aryl, —O—C(O)O-cycloalkyl, —O—C(O)O-substituted
cycloalkyl, —O—C(O)O-cycloalkenyl, —O—C(O)O-substituted
cycloalkenyl, —O—C(O)O-heteroaryl, —O—C(O)O-substituted heteroaryl, —O—C(O)O-heterocyclic,
   and —O—C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” refers to the group —CN.

“Cycloalkyl” refers to cyclic alkyl groups of from 3 to 10 carbon atoms having single or multiple cyclic rings including fused, bridged, and spiro ring systems. One or more of the rings can be aryl, heteroaryl, or heterocyclic provided that the point of attachment is through the non-aromatic, non-heterocyclic ring carbocyclic ring. Examples of suitable cycloalkyl groups include, for instance, adamantyl, cyclopropyl, cyclobutyl, cyclopentyl, and cyclooctyl. Other examples of cycloalkyl groups include bicycle[2,2,2,]octanyl, norbornyl, and spirobicyclo groups such as spiro[4.5]dec-8-yl.

“Cycloalkenyl” refers to non-aromatic cyclic alkyl groups of from 3 to 10 carbon atoms having single or multiple cyclic rings and having at least one >C═C< ring unsaturation and preferably from 1 to 2 sites of >C═C< ring unsaturation.

“Substituted cycloalkyl” and “substituted cycloalkenyl” refers to a cycloalkyl or cycloalkenyl group having from 1 to 5 or preferably 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Cycloalkyloxy” refers to —O-cycloalkyl.

“Substituted cycloalkyloxy” refers to —O-(substituted cycloalkyl).

“Cycloalkylthio” refers to —S-cycloalkyl.

“Substituted cycloalkylthio” refers to —S-(substituted cycloalkyl).

“Cycloalkenyloxy” refers to —O-cycloalkenyl.

“Substituted cycloalkenyloxy” refers to —O-(substituted cycloalkenyl).

“Cycloalkenylthio” refers to —S-cycloalkenyl.

“Substituted cycloalkenylthio” refers to —S-(substituted cycloalkenyl).

“Guanidino” refers to the group —NHC(═NH)NH₂.

“Substituted guanidino” refers to —NR⁴⁴C(═NR⁴⁴)N(R⁴⁴)₂ where each R⁴⁴ is independently selected from the group consisting of hydrogen, alkyl, substituted alkyl, aryl, substituted aryl, heteroaryl, substituted heteroaryl, heterocyclic, and substituted heterocyclic and two R⁴⁴ groups attached to a common guanidino nitrogen atom are optionally joined together with the nitrogen bound thereto to form a heterocyclic or substituted heterocyclic group, provided that at least one R⁴⁴ is not hydrogen, and wherein said substituents are as defined herein.

“Halo” or “halogen” refers to fluoro, chloro, bromo and iodo and preferably is fluoro or chloro.

“Haloalkyl” refers to alkyl groups substituted with 1 to 5, 1 to 3, or 1 to 2 halo groups, wherein alkyl and halo are as defined herein.

“Haloalkoxy” refers to alkoxy groups substituted with 1 to 5, 1 to 3, or 1 to 2 halo groups, wherein alkoxy and halo are as defined herein.

“Haloalkylthio” refers to alkylthio groups substituted with 1 to 5, 1 to 3, or 1 to 2 halo groups, wherein alkylthio and halo are as defined herein.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridyl, pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, and/or sulfonyl moieties. Preferred heteroaryls include pyridinyl, pyrrolyl, indolyl, thiophenyl, and furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heteroaryloxy” refers to —O-heteroaryl.

“Substituted heteroaryloxy” refers to the group —O-(substituted heteroaryl).

“Heteroarylthio” refers to the group —S-heteroaryl.

“Substituted heteroarylthio” refers to the group —S-(substituted heteroaryl).

“Heterocycle” or “heterocyclic” or “heterocycloalkyl” or “heterocyclyl” refers to a saturated or partially saturated, but not aromatic, group having from 1 to 10 ring carbon atoms and from 1 to 4 ring heteroatoms selected from the group consisting of nitrogen, sulfur, or oxygen. Heterocycle encompasses single ring or multiple condensed rings, including fused bridged and spiro ring systems. In fused ring systems, one or more the rings can be cycloalkyl, aryl, or heteroaryl provided that the point of attachment is through the non-aromatic heterocyclic ring. In one embodiment, the nitrogen and/or sulfur atom(s) of the heterocyclic group are optionally oxidized to provide for the N-oxide, sulfinyl, and/or sulfonyl moieties.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

“Heterocyclyloxy” refers to the group —O-heterocycyl.

“Substituted heterocyclyloxy” refers to the group —O-(substituted heterocycyl).

“Heterocyclylthio” refers to the group —S-heterocycyl.

“Substituted heterocyclylthio” refers to the group —S-(substituted heterocycyl).

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O) or (—O⁻).

“Spiro ring systems” refers to bicyclic ring systems that have a single ring carbon atom common to both rings.

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-cycloalkyl, —SO₂-substituted cycloalkyl, —SO₂-cycloalkenyl, —SO₂-substituted cycloalkenyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Substituted sulfonyl includes groups such as methyl-SO₂—, phenyl-SO₂—, and 4-methylphenyl-SO₂—. The term “alkylsulfonyl” refers to —SO₂-alkyl. The term “haloalkylsulfonyl” refers to —SO₂-haloalkyl where haloalkyl is defined herein. The term “(substituted sulfonyl)amino” refers to —NH(substituted sulfonyl), and the term “(substituted sulfonyl)aminocarbonyl” refers to —C(O)NH(substituted sulfonyl), wherein substituted sulfonyl is as defined herein.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cycloalkyl, —OSO₂-cycloalkenyl, —OSO₂-substituted cycloalkenyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, cycloalkenyl-C(S)—, substituted cycloalkenyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thiol” refers to the group —SH.

“Thiocarbonyl” refers to the divalent group —C(S)— which is equivalent to —C(═S)—.

“Thione” refers to the atom (═S).

“Alkylthio” refers to the group —S-alkyl wherein alkyl is as defined herein.

“Substituted alkylthio” refers to the group —S-(substituted alkyl) wherein substituted alkyl is as defined herein.

“Compound” or “compounds” as used herein is meant to include the stereoisomers and tautomers of the indicated formulas.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

As used herein, the term “phosphate ester” refers to any one of the mono-, di- or triphosphate esters of noribogaine, wherein the mono-, di- or triphosphate ester moiety is bonded to the 12-hydroxy group and/or the indole nitrogen of noribogaine.

As used herein, the term “phosphate ester” refers to any one of the mono-, di- or triphosphate esters of noribogaine, wherein the mono-, di- or triphosphate ester moiety is bonded to the 12-hydroxy group and/or the indole nitrogen of noribogaine.

As used herein, the term “monophosphate” refers to the group —P(O)(OH)₂.

As used herein, the term “diphosphate” refers to the group —P(O)(OH)—OP(O)(OH)₂.

As used herein, the term “triphosphate” refers to the group —P(O)(OH)—(OP(O)(OH))₂OH.

As used herein, the term “ester” as it refers to esters of the mono-, di- or triphosphate group means esters of the monophosphate can be represented by the formula —P(O)(OR⁴⁵)₂, where each R⁴⁵ is independently hydrogen, C₁-C₁₂ alkyl, C₃-C₁₀ cycloalkyl, C₆-C₁₄ aryl, heteroaryl of 1 to 10 carbon atoms and 1 to 4 optionally oxidized heteroatoms selected from the group consisting of oxygen, nitrogen, and sulfur and the like, provided that at least one R⁴⁵ is not hydrogen. Likewise, exemplary esters of the di- or triphosphate can be represented by the formulas —P(O)(OR⁴⁵)—OP(O)(OR⁴⁵)₂ and —P(O)(OR⁴⁵)—(OP(O)(OR⁴⁵))₂OR⁴⁵, where R⁴⁵ is as defined above.

As used herein, the term “hydrolyzable group” refers to a group that can be hydrolyzed to release the free hydroxy group under hydrolysis conditions. Examples of hydrolysable group include, but are not limited to those defined for R above. Preferred hydrolysable groups include carboxyl esters, phosphates and phosphate esters. The hydrolysis may be done by chemical reactions conditions such as base hydrolysis or acid hydrolysis or may be done in vivo by biological processes, such as those catalyzed by a phosphate hydrolysis enzyme. Nonlimiting examples of hydrolysable group include groups linked with an ester-based linker (—C(O)O— or —OC(O)—), an amide-based linker (—C(O)NR⁴⁶— or —NR⁴⁶C(O)—), or a phosphate-linker (—P(O)(OR⁴⁶)—O—, —O—P(S)(OR⁴⁶)—O—, —O—P(S)(SR⁴⁶)—O—, —S—P(O)(OR⁴⁶)—O—, —O—P(O)(OR⁴⁶)—S—, —S—P(O)(OR⁴⁶)—S—, —O—P(S)(OR⁴⁶)—S—, —S—P(S)(OR⁴⁶)—O—, —O—P(O)(OR⁴⁶)—O—, —O—P(S)(R⁴⁶)—O—, —S—P(O)(R⁴⁶)—O—, —S—P(S)(R⁴⁶)—O—, —S—P(O)(R⁴⁶)—S—, or —O—P(S)(R⁴⁶)—S—) where R⁴⁶ can be hydrogen or alkyl.

Substituted groups of this invention, as set forth above, do not include polymers obtained by an infinite chain of substituted groups. At most, any substituted group can be substituted up to five times.

“Noribogaine” refers to the compound:

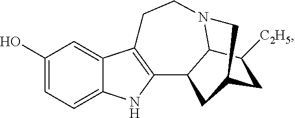

as well as noribogaine derivatives or pharmaceutically acceptable salts and pharmaceutically acceptable solvates thereof. It should be understood that where “noribogaine” is mentioned herein, one more polymorphs of noribogaine can be utilized and are contemplated. In some embodiments, noribogaine is noribogaine glucuronide. Noribogaine can be prepared by demethylation of naturally occurring ibogaine:

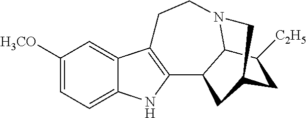

which is isolated from Tabernanth iboga, a shrub of West Africa. Demethylation may be accomplished by conventional techniques such as by reaction with boron tribromide/methylene chloride at room temperature followed by conventional purification. See, for example, Huffman, et al., J. Org. Chem. 50:1460 (1985), which incorporated herein by reference in its entirety.

Noribogaine can be synthesized as described, for example in U.S. Patent Pub. Nos. 2013/0165647, 2013/0303756, and 2012/0253037, PCT Patent Publication No. WO 2013/040471 (includes description of making noribogaine polymorphs), and U.S. patent application Ser. No. 13/593,454, each of which is incorporated herein by reference in its entirety.

“Noribogaine derivatives” refer, without limitation, to esters or O-carbamates of noribogaine, or pharmaceutically acceptable salts and/or solvates of each thereof. Also encompassed within this invention are derivatives of noribogaine that act as prodrug forms of noribogaine. A prodrug is a pharmacological substance administered in an inactive (or significantly less active) form. Once administered, the prodrug is metabolized in vivo into an active metabolite. Noribogaine derivatives include, without limitation, those compounds set forth in U.S. Pat. Nos. 6,348,456 and 8,362,007; as well as in U.S. patent application Ser. No. 13/165,626; and US Patent Application Publication Nos. US2013/0131046; US2013/0165647; US2013/0165425; and US2013/0165414; all of which are incorporated herein by reference. Non-limiting examples of noribogaine derivatives encompassed by this invention are given in more detail in the “Compositions” section below.

In some embodiments, the methods of the present disclosure entail the administration of a prodrug of noribogaine that provides the desired maximum serum concentrations and efficacious average noribogaine serum levels. A prodrug of noribogaine refers to a compound that metabolizes, in vivo, to noribogaine. In some embodiments, the prodrug is selected to be readily cleavable either by a cleavable linking arm or by cleavage of the prodrug entity that binds to noribogaine such that noribogaine is generated in vivo. In one preferred embodiment, the prodrug moiety is selected to facilitate binding to the μ opioid and/or κ opioid receptors in the brain either by facilitating passage across the blood brain barrier or by targeting brain receptors other than the μ and/or κ opioid receptors. Examples of prodrugs of noribogaine are provided in U.S. patent application Ser. No. 13/165,626, the entire content of which is incorporated herein by reference.

This invention is not limited to any particular chemical form of noribogaine or noribogaine derivative, and the drug may be given to patients either as a free base, solvate, or as a pharmaceutically acceptable acid addition salt. In the latter case, the hydrochloride salt is generally preferred, but other salts derived from organic or inorganic acids may also be used. Examples of such acids include, without limitation, those described below as “pharmaceutically acceptable salts” and the like.

“Pharmaceutically acceptable composition” refers to a composition that is suitable for administration to a mammal, preferably a human. Such compositions include various excipients, diluents, carriers, and such other inactive agents well known to the skilled artisan.

“Pharmaceutically acceptable salt” refers to pharmaceutically acceptable salts, including pharmaceutically acceptable partial salts, of a compound, which salts are derived from a variety of organic and inorganic counter ions well known in the art and include, by way of example only, hydrochloric acid, hydrobromic acid, phosphoric acid, sulfuric acid, methane sulfonic acid, phosphorous acid, nitric acid, perchloric acid, acetic acid, tartaric acid, lactic acid, succinic acid, citric acid, malic acid, maleic acid, aconitic acid, salicylic acid, thalic acid, embonic acid, enanthic acid, oxalic acid and the like, and when the molecule contains an acidic functionality, include, by way of example only, sodium, potassium, calcium, magnesium, ammonium, tetraalkylammonium, and the like.

A “pharmaceutically acceptable solvate” or “hydrate” of a compound of the invention means a solvate or hydrate complex that is pharmaceutically acceptable and that possesses the desired pharmacological activity of the parent compound, and includes, but is not limited to, complexes of a compound of the invention with one or more solvent or water molecules, or 1 to about 100, or 1 to about 10, or one to about 2, 3 or 4, solvent or water molecules.

“Therapeutically effective amount” or “therapeutic amount” refers to an amount of a drug or an agent that, when administered to a patient suffering from a condition, will have the intended therapeutic effect, e.g., alleviation, amelioration, palliation or elimination of one or more manifestations of the condition in the patient. The therapeutically effective amount will vary depending upon the patient and the condition being treated, the weight and age of the subject, the severity of the condition, the salt, solvate, or derivative of the active drug portion chosen, the particular composition or excipient chosen, the dosing regimen to be followed, timing of administration, the manner of administration and the like, all of which can be determined readily by one of ordinary skill in the art. The full therapeutic effect does not necessarily occur by administration of one dose, and may occur only after administration of a series of doses. Thus, a therapeutically effective amount may be administered in one or more administrations. For example, and without limitation, a therapeutically effective amount of noribogaine, in the context of treating drug dependency, refers to an amount of noribogaine that attenuates the dependency and/or symptoms of acute withdrawal for at least 2 hours beyond control (placebo), at least 5 hours beyond control, and preferably at least 10 hours beyond control.

A “therapeutic level” or “therapeutic serum concentration” of a drug is an amount of noribogaine, noribogaine derivative, or pharmaceutical salt or solvate thereof that is sufficient to treat a condition or to treat, prevent, or attenuate acute symptoms thereof, but not high enough to pose any significant risk to the patient. Therapeutic levels of drugs can be determined by tests that measure the actual concentration of the compound in the blood of the patient. This concentration is referred to as the “serum concentration.” Where the serum concentration of noribogaine is mentioned, it is to be understood that the term “noribogaine” encompasses any form of noribogaine, including derivatives thereof.

The therapeutically effective amount of the compound may be higher or lower, depending on the route of administration used. For example, when direct blood administration (e.g., sublingual, pulmonary and intranasal delivery) is used, a lower dose of the compound may be administered. In one aspect, a therapeutically effective amount of noribogaine or derivative is from about 50 ng to less than 100 μg per kg of body weight. Where other routes of administration are used, a higher dose of the compound may be administered. In one embodiment, the therapeutically effective amount of the compound is from greater than about 1 mg to about 8 mg per kg of body weight per day.

A “sub-therapeutic level” of noribogaine or pharmaceutical salt and/or solvate thereof that is less than the therapeutic level described above. For example, the sub-therapeutic level of noribogaine may be e.g., 80%, 70%, 60%, 50%, 40%, 30%, 20%, or 10% less than a therapeutically effective amount (e.g., 120 mg) of noribogaine, or any subvalue or subrange there between. Sub-therapeutic levels of noribogaine may coincide with “maintenance amounts” of noribogaine which are amounts, less than the therapeutically effective amount, that provide some attenuation and/or prevention of post-acute withdrawal syndrome in a patient. The maintenance amount of the compound is expected to be less than the therapeutically effective amount.

As defined herein, a “prophylactically effective amount” of a drug is an amount, typically less than the therapeutically effective amount, that provides attenuation and/or prevention of a disease or disorder or symptoms of a disease or disorder in a patient. For example, the prophylactically effective amount of the compound is expected to be less than the therapeutically effective amount because the level of inhibition does not need to be as high in a patient who no longer has a disease or disorder or symptoms of a disease or disorder (e.g., no longer physically addicted to nicotine). For example, a prophylactically effective amount is preferably 90%, 80%, 70%, 60%, 50%, 40%, 30%, 20%, or 10% less than a therapeutically effective amount. However, a prophylactically effective amount may be the same as the therapeutically effective amount, for example when a patient who is physically addicted to nicotine is administered noribogaine to attenuate cravings for a period of time when nicotine use is not feasible. The prophylactically effective amount may vary for different a diseases or disorders or symptoms of different diseases or disorders.

As defined herein, a “maintenance amount” of a drug or an agent is an amount, typically less than the therapeutically effective amount that provides attenuation and/or prevention of syndrome disease or disorder or symptoms of a disease or disorder in a patient. The maintenance amount of the compound is expected to be less than the therapeutically effective amount because the level of inhibition does not need to be as high in a patient who is no longer physically manifests a disease or disorder or symptoms of a disease or disorder. For example, a maintenance amount is preferably 90%, 80%, 70%, 60%, 50%, 40%, 30%, 20%, or 10% less than a therapeutically effective amount, or any subvalue or subrange there between.

“Treatment,” “treating,” and “treat” are defined as acting upon a disease, disorder, or condition with an agent, such as noribogaine, to reduce or ameliorate harmful or any other undesired effects of the disease, disorder, or condition and/or its symptoms. “Treatment,” as used herein, covers the treatment of a human patient, and includes: (a) reducing the risk of occurrence of the condition in a patient determined to be predisposed to the condition but not yet diagnosed as having the condition, (b) impeding the development of the condition, and/or (c) relieving the condition, i.e., causing regression of the condition and/or relieving one or more symptoms of the condition. “Treating” or “treatment of” a condition or patient refers to taking steps to obtain beneficial or desired results, including clinical results such as the reduction of symptoms. For example, with regard to drug addiction, beneficial or desired clinical results include, but are not limited to: treating drug addiction; treating, preventing, and/or attenuating acute withdrawal symptoms; treating, preventing, and/or attenuating long-term (post-acute) withdrawal symptoms; and preventing relapse of drug use.

As used herein, the term “patient” refers to mammals and includes humans and non-human mammals.

As used herein, the term “QT interval” refers to the measure of the time between the start of the Q wave and the end of the T wave in the electrical cycle of the heart. Prolongation of the QT interval refers to an increase in the QT interval.

As used herein the term “solvate” is taken to mean that a solid-form of a compound that crystallizes with one or more molecules of solvent trapped inside. A few examples of solvents that can be used to create solvates, such as pharmaceutically acceptable solvates, include, but are certainly not limited to, water, methanol, ethanol, isopropanol, butanol, C₁-C₆ alcohols in general (and optionally substituted), tetrahydrofuran, acetone, ethylene glycol, propylene glycol, acetic acid, formic acid, water, and solvent mixtures thereof. Other such biocompatible solvents which may aid in making a pharmaceutically acceptable solvate are well known in the art and applicable to the present invention. Additionally, various organic and inorganic acids and bases can be added or even used alone as the solvent to create a desired solvate. Such acids and bases are known in the art. When the solvent is water, the solvate can be referred to as a hydrate. Further, by being left in the atmosphere or recrystallized, the compounds of the present invention may absorb moisture, may include one or more molecules of water in the formed crystal, and thus become a hydrate. Even when such hydrates are formed, they are included in the term “solvate”. Solvate also is meant to include such compositions where another compound or complex co-crystallizes with the compound of interest. The term “solvate” as used herein refers to complexes with solvents in which noribogaine is reacted or from which noribogaine is precipitated or crystallized. For example, a complex with water is known as a “hydrate”. Solvates of noribogaine are within the scope of the invention. It will be appreciated by those skilled in organic chemistry that many organic compounds can exist in more than one crystalline form. For example, crystalline form may vary based on the solvate used. Thus, all crystalline forms of noribogaine or the pharmaceutically acceptable solvates thereof are within the scope of the present invention.

The term “dose” refers to a range of noribogaine, noribogaine derivative, or pharmaceutical salt or solvate thereof that provides a therapeutic serum level of noribogaine when given to a patient in need thereof. The dose is recited in a range, for example from about 20 mg to about 120 mg, and can be expressed either as milligrams or as mg/kg body weight. The attending clinician will select an appropriate dose from the range based on the patient's weight, age, degree of addiction, health, and other relevant factors, all of which are well within the skill of the art.

The term “unit dose” refers to a dose of drug that is given to the patient to provide therapeutic results, independent of the weight of the patient. In such an instance, the unit dose is sold in a standard form (e.g., 20 mg tablet). The unit dose may be administered as a single dose or a series of subdoses. In some embodiments, the unit dose provides a standardized level of drug to the patient, independent of weight of patient. Many medications are sold based on a dose that is therapeutic to all patients based on a therapeutic window. In such cases, it is not necessary to titrate the dosage amount based on the weight of the patient.

### II. Compositions

As will be apparent to the skilled artisan upon reading this disclosure, this invention provides compositions comprising noribogaine, noribogaine derivative, prodrugs of noribogaine, pharmaceutically acceptable salts and/or solvates of each thereof. This invention further provides compositions for achieving and maintaining a therapeutic serum concentration of the drug in a patient.

As will be apparent to the skilled artisan upon reading this disclosure, this invention provides compositions for treating or preventing diseases or disorders or symptoms of diseases or disorders described herein, comprising noribogaine, noribogaine derivatives, prodrugs of noribogaine, pharmaceutically acceptable salts and/or solvates of each thereof. This invention further provides compositions for treating, attenuating, or preventing symptoms of diseases or disorders described herein, comprising noribogaine, noribogaine derivatives, prodrugs of noribogaine, pharmaceutically acceptable salts and/or solvates of each thereof.

In some embodiments, the composition is formulated for oral, transdermal, internal, pulmonary, rectal, nasal, vaginal, lingual, intravenous, intraarterial, intramuscular, intraperitoneal, intracutaneous or subcutaneous delivery. In one embodiment, the therapeutically effective amount of the compound is from about 1 mg to about 4 mg per kg body weight per day. In another embodiment, the therapeutically effective amount of the compound is from about 1 mg to about 3 mg per kg body weight per day. In another embodiment, the therapeutically effective amount of the compound is from about 1 mg to about 2 mg per kg body weight per day. In another embodiment, the therapeutically effective amount of the compound is from about 1.3 mg to about 3 mg per kg body weight per day. In another embodiment, the therapeutically effective amount of the compound is from about 1.5 mg to about 3 mg per kg body weight per day. In another embodiment, the therapeutically effective amount of the compound is from about 1.3 mg to about 4 mg per kg body weight per day. In another embodiment, the therapeutically effective amount of the compound is from about 1.5 mg to about 4 mg per kg body weight per day. The ranges include both extremes as well as any subranges there between.

In one embodiment, the therapeutically effective amount of the compound is from about 1 mg to about 8 mg per kg body weight per day. In another embodiment, the therapeutically effective amount of the compound is from about 1.3 mg to about 7 mg per kg body weight per day. In another embodiment, the therapeutically effective amount of the compound is from about 1.3 mg to about 6 mg per kg body weight per day. In another embodiment, the therapeutically effective amount of the compound is from about 1.3 mg to about 5 mg per kg body weight per day. In another embodiment, the therapeutically effective amount of the compound is from about 1.3 mg to about 4 mg per kg body weight per day. In another embodiment, the therapeutically effective amount of the compound is from about 1.3 mg to about 2 mg per kg body weight per day. In another embodiment, the therapeutically effective amount of the compound is from about 1.5 mg to about 3 mg per kg body weight per day. In another embodiment, the therapeutically effective amount of the compound is from about 1.7 mg to about 3 mg per kg body weight per day. In another embodiment, the therapeutically effective amount of the compound is from about 2 mg to about 4 mg per kg body weight per day. In another embodiment, the therapeutically effective amount of the compound is from about 2 mg to about 3 mg per kg body weight per day.

In one embodiment, the therapeutically effective amount of the compound is about 8 mg/kg body weight per day. In one embodiment, the therapeutically effective amount of the compound is about 7 mg/kg body weight per day. In one embodiment, the therapeutically effective amount of the compound is about 6 mg/kg body weight per day. In one embodiment, the therapeutically effective amount of the compound is about 5 mg/kg body weight per day. In one embodiment, the therapeutically effective amount of the compound is about 4 mg/kg body weight per day. In one embodiment, the therapeutically effective amount of the compound is about 3 mg/kg body weight per day. In one embodiment, the therapeutically effective amount of the compound is about 2 mg/kg body weight per day. In one embodiment, the therapeutically effective amount of the compound is about 1 mg/kg body weight per day.

In one embodiment, the therapeutically effective amount of the compound is about 4 mg/kg body weight per day. In one embodiment, the therapeutically effective amount of the compound is about 3 mg/kg body weight per day. In another embodiment, the therapeutically effective amount of the compound is about 2 mg per kg body weight per day. In another embodiment, the therapeutically effective amount of the compound is about 1.7 mg per kg body weight per day. In another embodiment, the therapeutically effective amount of the compound is about 1.5 mg per kg body weight per day. In another embodiment, the therapeutically effective amount of the compound is about 1.2 mg per kg body weight per day. In another embodiment, the therapeutically effective amount of the compound is about 1 mg per kg body weight per day.

In one aspect, the invention provides a pharmaceutical composition comprising a therapeutically or prophylactically effective amount of noribogaine and a pharmaceutically acceptable excipient, wherein the therapeutically or prophylactically effective amount of noribogaine is an amount that delivers an aggregate amount of noribogaine of about 50 ng to less than 10 μg per kg body weight per day. In some aspects, the therapeutically or prophylactically effective amount of noribogaine is an amount that delivers an aggregate amount of noribogaine of about 50 ng to about 5 μg per kg body weight per day. In some aspects, the therapeutically or prophylactically effective amount of noribogaine is an amount that delivers an aggregate amount of noribogaine of about 50 ng to about 1 μg per kg body weight per day. In some aspects, the composition is formulated for administration once per day. In some aspects, the composition is formulated for administration two or more times per day.

In some embodiments, the composition is formulated for sublingual, intranasal, or intrapulmonary delivery. In one aspect, the invention provides a pharmaceutical composition comprising a pharmaceutically effective amount of noribogaine and a pharmaceutically acceptable excipient, wherein the therapeutically effective amount of noribogaine is an amount that delivers an aggregate amount of noribogaine of 50 ng to less than 100 μg per kg body weight per day. In some aspects, the therapeutically effective amount of noribogaine is an amount that delivers an aggregate amount of noribogaine of 50 ng to 50 μg per kg body weight per day. In some aspects, the therapeutically effective amount of noribogaine is an amount that delivers an aggregate amount of noribogaine of 50 ng to 10 μg per kg body weight per day. In some aspects, the therapeutically effective amount of noribogaine is an amount that delivers an aggregate amount of noribogaine of 50 ng to 1 μg per kg body weight per day. In some aspects, the composition is formulated for administration once per day. In some aspects, the composition is formulated for administration two or more times per day. The ranges include both extremes as well as any subranges there between.

In another embodiment, the therapeutically effective amount of the compound is from 1.3 mg to 4 mg per kg body weight per day. In another embodiment, the therapeutically effective amount of the compound is from 1.5 mg to 3 mg per kg body weight per day. In another embodiment, the therapeutically effective amount of the compound is from 1.7 mg to 3 mg per kg body weight per day. In another embodiment, the therapeutically effective amount of the compound is from 2 mg to 4 mg per kg body weight per day. In another embodiment, the therapeutically effective amount of the compound is from 2 mg to 3 mg per kg body weight per day.

### Compounds Utilized

In one embodiment, the noribogaine derivative is represented by Formula I:

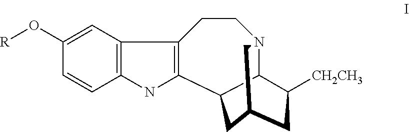

or a pharmaceutically acceptable salt and/or solvate thereof,
wherein R is hydrogen or a hydrolyzable group such as hydrolyzable esters of from about 1 to 12 carbons.

Generally, in the above formula, R is hydrogen or a group of the formula:

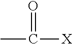

wherein X is a C₁-C₁₂ group, which is unsubstituted or substituted. For example, X may be a linear alkyl group such as methyl, ethyl, n-propyl, n-butyl, n-pentyl, n-hexyl, n-heptyl, n-octyl, n-nonyl, n-decyl, n-undecyl or n-dodecyl, or a branched alkyl group, such as i-propyl or sec-butyl. Also, X may be a phenyl group or benzyl group, either of which may be substituted with lower alkyl groups or lower alkoxy groups. Generally, the lower alkyl and/or alkoxy groups have from 1 to about 6 carbons. For example, the group R may be acetyl, propionyl or benzoyl. However, these groups are only exemplary.

Generally, for all groups X, they may either be unsubstituted or substituted with lower alkyl or lower alkoxy groups. For example, substituted X may be o-, m- or p-methyl or methoxy benzyl groups.

C₁-C₁₂ groups include C₁-C₁₂ alkyl, C₃-C₁₂ cycloalkyl, C₆-C₁₂ aryl, C₇-C₁₂ arylalkyl, wherein Cₓ indicates that the group contains x carbon atoms. Lower alkyl refers to C₁-C₄ alkyl and lower alkoxy refers to C₁-C₄ alkoxy.

In one embodiment, the noribogaine derivative is represented by Formula II:

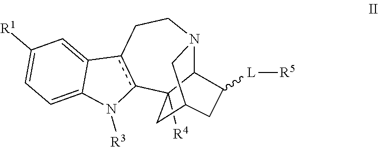

or a pharmaceutically acceptable salt and/or solvate thereof,
wherein
[symbol] is a single or double bond;
R¹ is halo, OR², or C₁-C₁₂ alkyl optionally substituted with 1 to 5 R¹⁰; R² is hydrogen or a hydrolysable group selected from the group consisting of —C(O)R^{x}, —C(O)OR^{x} and —C(O)N(R^{Y})₂ where each R^{x} is selected from the group consisting of C₁-C₆ alkyl optionally substituted with 1 to 5 R¹⁰, and each R^{Y} is independently selected from the group consisting of hydrogen, C₁-C₆ alkyl optionally substituted with 1 to 5 R¹⁰, C₆-C₁₄ aryl optionally substituted with 1 to 5 R¹⁰, C₃-C₁₀ cycloalkyl optionally substituted with 1 to 5 R¹⁰, C₁-C₁₀ heteroaryl having 1 to 4 heteroatoms and which is optionally substituted with 1 to 5 R¹⁰, C₁-C₁₀ heterocyclic having 1 to 4 heteroatoms and which is optionally substituted with 1 to 5 R¹⁰, and where each R^{y}, together with the nitrogen atom bound thereto form a C₁-C₆ heterocyclic having 1 to 4 heteroatoms and which is optionally substituted with 1 to 5 R¹⁰ or a C₁-C₆ heteroaryl having 1 to 4 heteroatoms and which is optionally substituted with 1 to 5 R¹⁰;
R³ is selected from the group consisting of hydrogen, C₁-C₂ alkyl optionally substituted with 1 to 5 R¹⁰, aryl optionally substituted with 1 to 5 R¹⁰, —C(O)R⁶, —C(O)NR⁶R⁶ and —C(O)OR⁶;
R⁴ is selected from the group consisting of hydrogen, —(CH₂)ₘOR⁸, —CR⁷(OH)R⁸, —(CH₂)ₘCN, —(CH₂)ₘCOR, —(CH₂)ₘCO₂R⁸, —(CH₂)ₘC(O)NR⁷R⁸, —(C H₂)ₘC(O)NR⁷NR⁸R⁸, —(CH₂)ₘC(O)NR⁷NR⁸C(O)R⁹, and —(CH₂)ₘNR⁷R⁸;
m is 0, 1, or 2;
L is a bond or C₁-C₁₂ alkylene;
R⁵ is selected from the group consisting of hydrogen, C₁-C₁₂ alkyl substituted with 1 to 5 R¹⁰, C₁-C₁₂ alkenyl substituted with 1 to 5 R¹⁰, —X¹—R⁷, —(X¹—Y)ₙ—X¹—R⁷, —SO₂NR⁷R⁸, —O—C(O)R⁹, —C(O)OR⁸, —C(O)NR⁷R⁸, —NR⁷R⁸, —NHC(O)R⁹, and —NR⁷C(O)R⁹;
each R⁶ is independently selected from the group consisting of hydrogen, C₁-C₁₂ alkyl, C₂-C₁₂ alkenyl, C₂-C₁₂ alkynyl, C₆-C₁₀ aryl, C₁-C₆ heteroaryl having 1 to 4 heteroatoms, and C₁-C₆ heterocycle having 1 to 4 heteroatoms, and wherein the alkyl, alkenyl, alkynyl, aryl, heteroaryl, and heterocycle are optionally substituted with 1 to 5 R¹⁰;
X is selected from the group consisting of O and S;
Y is C₁-C₄ alkylene or C₆-C₁₀ arylene, or a combination thereof;
n is 1, 2, or 3;
R⁷ and R⁸ are each independently selected from the group consisting of hydrogen, C₁-C₁₂ alkyl optionally substituted with 1 to 5 R¹⁰, C₁-C₆ heterocycle having 1 to 4 heteroatoms and which is optionally substituted with 1 to 5 R¹⁰, C₃-C₁₀ cycloalkyl optionally substituted with 1 to 5 R¹⁰, C₆-C₁₀ aryl optionally substituted with 1 to 5 R¹⁰ and C₁-C₆ heteroaryl having 1 to 4 heteroatoms optionally substituted with 1 to 5 R¹⁰;
R⁹ is selected from the group consisting of C₁-C₁₂ alkyl optionally substituted with 1 to 5 R¹⁰, C₁-C₆ heterocycle having 1 to 4 heteroatoms optionally substituted with 1 to 5 R¹⁰, C₃-C₁₀ cycloalkyl optionally substituted with 1 to 5 R¹⁰, C₆-C₁₀ aryl optionally substituted with 1 to 5 R¹⁰ and C₁-C₆ heteroaryl having 1 to 4 heteroatoms optionally substituted with 1 to 5 R¹⁰;
R¹⁰ is selected from the group consisting of C₁-C₄ alkyl, phenyl, halo, —OR¹¹, —CN, —COR¹¹, —CO₂R¹¹, —C(O)NHR¹¹, —NR¹¹R¹¹, —C(O)NR¹¹R¹¹, —C(O)NHNHR¹¹, —C(O)NR¹¹NHR¹¹, —C(O)NR¹¹NR¹¹R¹¹, —C(O)NHNR¹¹C(O)R¹¹, —C(O)NHNHC(O) R¹¹, —SO₂NR¹¹R¹¹, —C(O)NR¹¹NR¹¹C(O)R¹¹, and —C(O)NR¹¹NHC(O)R¹¹; and R¹¹ is independently hydrogen or C₁-C₁₂ alkyl;
provided that:
when L is a bond, then R⁵ is not hydrogen;
when [symbol] is a double bond, R¹ is an ester hydrolyzable group, R³ and R⁴ are both hydrogen, then -L-R⁵ is not ethyl;
when [symbol] is a double bond, R¹ is —OH, halo or C₁-C₁₂ alkyl optionally substituted with 1 to 5 R¹⁰, then R⁴ is hydrogen; and
when [symbol] is a double bond, R is OR², R⁴ is hydrogen, -L-R⁵ is ethyl, then R² is not a hydrolyzable group selected from the group consisting of an ester, amide, carbonate and carbamate.

In one embodiment, the noribogaine derivative is represented by Formula III:

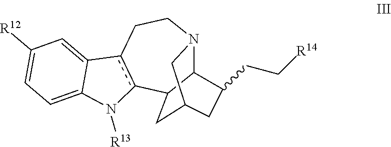

or a pharmaceutically acceptable salt and/or solvate thereof,
wherein
[symbol] a single or double bond;
R¹² is halo, —OH, —SH, —NH₂, —S(O)₂N(R¹⁷)₂, —R^{z}-L¹-R¹⁸, —R^{z}-L¹-R¹⁹, —R^{z}-L¹-R²⁰ or —R^{z}- L¹-CHR¹⁸R¹⁹, where R^{z} is O, S or NR¹⁷;
L¹ is alkylene, arylene, —C(O)-alkylene, —C(O)-arylene, —C(O)O-arylene, —C(O)O— alkylene, —C(O)NR²⁰-alkylene, —C(O)NR²⁰-arylene, —C(NR²⁰)NR²⁰-alkylene or —C(NR²⁰)NR²⁰-arylene, wherein L¹ is configured such that —O-L¹-R¹⁸ is —OC(O)-alkylene-R¹⁸, —OC(O)O-arylene-R¹⁸, —OC(O)O-alkylene-R¹⁸, —OC(O)-arylene-R¹⁸, —OC(O)NR²⁰-alkylene-R¹⁸, —OC(O)NR²⁰-arylene-R¹⁸, —OC(NR²⁰)NR²⁰-alkylene-R¹⁸ or —OC(NR²⁰)NR²⁰-arylene-R¹⁸, and wherein the alkylene and arylene are optionally substituted with 1 to 2 R¹⁶;
R¹³ is hydrogen, —S(O)₂OR²⁰, —S(O)₂R²⁰, —C(O)R¹⁵, —C(O)NR¹⁵R¹⁵, —C(O)OR¹⁵, C₁-C₁₂ alkyl optionally substituted with 1 to 5 R¹⁶, C₁-C₁₂ alkenyl optionally substituted with 1 to 5 R¹⁶, or aryl optionally substituted with 1 to 5 R¹⁶;
R¹⁴ is hydrogen, halo, —OR¹⁷, —CN, C₁-C₁₂ alkyl, C₁-C₁₂ alkoxy, aryl or aryloxy, where the alkyl, alkoxy, aryl, and aryloxy are optionally substituted with 1 to 5 R¹⁶;
each R¹⁵ is independently selected from the group consisting of hydrogen, C₁-C₁₂ alkyl, C₂-C₁₂ alkenyl, C₂-C₁₂ alkynyl, aryl, heteroaryl, and heterocycle, and wherein the alkyl, alkenyl, alkynyl, aryl, heteroaryl, and heterocycle are optionally substituted with 1 to 5 R¹⁶;
R¹⁶ is selected from the group consisting of phenyl, halo, —OR¹⁷, —CN, —COR¹⁷, —CO₂R¹⁷, —NR¹⁷R¹⁷, —NR¹⁷C(O)R¹⁷, —NR¹⁷SO₂R¹⁷, —C(O)NR¹⁷R¹⁷, —C (O)NR¹⁷NR¹⁷R¹⁷, —SO₂NR¹⁷R¹⁷ and —C(O)NR¹⁷NR¹⁷C(O)R¹⁷;
each R¹⁷ is independently hydrogen or C₁-C₁₂ alkyl optionally substituted with from 1 to 3 halo;
R¹⁸ is hydrogen, —C(O)R²⁰, —C(O)OR²⁰, —C(O)N(R²⁰)₂ or —N(R²⁰)C(O)R²⁰;
R¹⁹ is hydrogen, —N(R²⁰)₂, —C(O)N(R²⁰)₂, —C(NR²⁰)N(R²⁰)₂, —C(NSO₂R²⁰)N(R²⁰)₂, —NR²⁰C(O)N(R²⁰)₂, —NR²⁰C(S)N(R²⁰)₂, —NR²⁰C(NR²⁰)N(R²⁰)₂, —NR²⁰C(NSO₂R²⁰)N(R²⁰)₂ or tetrazole; and
each R²⁰ is independently selected from the group consisting of hydrogen, C₁-C₁₂ alkyl and aryl;
provided that:
when [symbol] is a double bond and R¹³ and R¹⁴ are hydrogen, then R¹² is not hydroxy;
when [symbol] is a double bond, R¹⁴ is hydrogen, R¹² is —O-L¹-R¹⁸, —O-L¹-R¹⁹, —O-L¹-R²⁰, and L¹ is alkylene, then —O-L¹-R¹⁸, —O-L¹-R¹⁹, —O-L¹-R²⁰ are not methoxy;
when [symbol] is a double bond, R¹⁴ is hydrogen, R^{z} is O, L¹ is —C(O)-alkylene, —C(O)-arylene, —C(O)O-arylene, —C(O)O-alkylene, —C(O)NR²⁰-alkylene, or —C(O)NR²⁰-arylene, then none of R¹⁸, R¹⁹ or R²⁰ are hydrogen.

In one embodiment, the noribogaine derivative is represented b Formula IV:

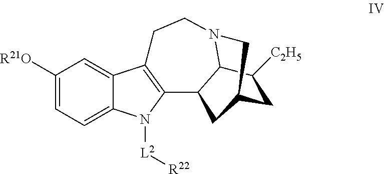

or a pharmaceutically acceptable salt and/or solvate thereof,

wherein

R²¹ is selected from the group consisting of hydrogen, a hydrolysable group selected from the group consisting of —C(O)R²³, —C(O)NR²⁴R²⁵ and —C(O)OR²⁶, where R²³ is selected from the group consisting of hydrogen, alkyl, substituted alkyl, alkenyl, substituted alkenyl, alkynyl and substituted alkynyl, R²⁴ and R²⁵ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic, R²⁶ is selected from the group consisting of alkyl, substituted alkyl, alkenyl, substituted alkenyl, alkynyl, substituted alkynyl, aryl, substituted aryl, heteroaryl, substituted heteroaryl, heterocyclic and substituted heterocyclic, provided that R²¹ is not a saccharide or an oligosaccharide;

L² is selected from the group consisting of a covalent bond and a cleavable linker group;

R²² is selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic, provided that R is not a saccharide or an oligosaccharide;

provided that when L² is a covalent bond and R²² is hydrogen, then R²¹ is selected from the group consisting of —C(O)NR²⁴R²⁵ and —C(O)OR²⁶; and

further provided that when R²¹ is hydrogen or —C(O)R²³ and L² is a covalent bond, then R²² is not hydrogen.

In one embodiment, the noribogaine derivative is represented by Formula V:

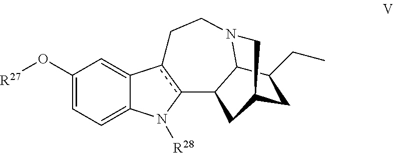

or a pharmaceutically acceptable salt and/or solvate thereof,

wherein:

[symbol] refers to a single or a double bond provided that when [symbol] is a single bond, Formula V refers to the corresponding dihydro compound;

R²⁷ is hydrogen or SO₂OR²⁹;

R²⁸ is hydrogen or SO₂OR²⁹;

R²⁹ is hydrogen or C₁-C₆ alkyl;

provided that at least one of R²⁷ and R²⁸ is not hydrogen.

In one embodiment, the noribogaine derivative is represented by Formula VI:

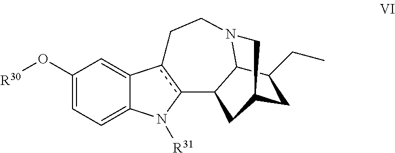

or a pharmaceutically acceptable salt and/or solvate thereof,
wherein:

[symbol] refers to a single or a double bond provided that when [symbol] is a single bond, Formula VI refers to the corresponding vicinal dihydro compound;

R³⁰ is hydrogen, a monophosphate, a diphosphate or a triphosphate; and

R³¹ is hydrogen, a monophosphate, a diphosphate or a triphosphate;

provided that both R³⁰ and R³¹ are not hydrogen;

wherein one or more of the monophosphate, diphosphate and triphosphate groups of R³⁰ and R³⁰ are optionally esterified with one or more C₁-C₆ alkyl esters.

Noribogaine as utilized herein, can be replaced by a noribogaine derivative or a salt of noribogaine or the noribogaine derivative or a solvate of each of the foregoing.

In a preferred embodiment, the compound utilized herein is noribogaine or a salt thereof. In a more preferred embodiment, the compound utilized herein is noribogaine.

### III. Methods of the Invention

As will be apparent to the skilled artisan upon reading this disclosure, one aspect of the present invention provides a method for treating a condition in a patient, such condition being treatable by noribogaine, noribogaine derivative, or pharmaceutically acceptable salt or solvate thereof, the method comprising administering to the patient an initial unit dose of noribogaine, noribogaine derivative, or pharmaceutically acceptable salt or solvate thereof, followed by at least one additional dose of noribogaine, noribogaine derivative, or pharmaceutically acceptable salt or solvate thereof, such that an average therapeutic serum concentration is achieved by the initial unit dose and maintained by the at least one additional dose.

Conditions that are treatable by noribogaine, noribogaine derivative, or pharmaceutically acceptable salt or solvate thereof include, without limitation, drug dependency and withdrawal (including opioids, nicotine, caffeine, alcohol, cocaine, barbiturates, narcotics, and other addictive drugs, both legal and illicit), depression, anxiety, post-traumatic stress disorder (PTSD), pain, opioid analgesic tolerance, and the like. Exemplary conditions are described, for example, in U.S. Provisional Patent Application Ser. Nos. 61/941,390; 61/952,731; 61/952,727; 61/952,733; 61/952,738; and 61/952,741; U.S. patent application Ser. Nos. 14/214,157; 14/346,655; 14/195,822; and 14/292,632. Each of these applications is incorporated herein by reference in its entirety.

In one aspect, this invention relates to a method for treating a condition in a patient which is treatable with noribogaine, noribogaine derivative, or pharmaceutically acceptable salt or solvate thereof while maintaining an acceptable QT interval prolongation in said patient, the method comprising:

a) administering to the patient an initial unit dose of noribogaine, noribogaine derivative, or pharmaceutically acceptable salt or solvate thereof, wherein said unit dose provides a therapeutic average serum concentration of 50 ng/mL to 180 ng/mL which serum concentration imparts minimal QT interval prolongation; and

b) maintaining said serum concentration by periodically administering at least one additional dose of noribogaine, noribogaine derivative, or pharmaceutically acceptable salt or solvate thereof, such that the at least one additional dose maintains the average serum concentration of 50 ng/mL to 180 ng/mL during treatment

wherein said additional dose or doses are continued as necessary to treat said condition.

In one aspect, the serum concentration provides a maximum QT interval of less than about 500 ms during said treatment. In some embodiments, the serum concentration provides a maximum QT interval of less than about 470 ms during treatment. Preferably, the serum concentration provides a maximum QT interval of less than about 450 ms during treatment. In one embodiment, the serum concentration provides a maximum QT interval of less than about 420 ms during treatment.

In one embodiment, the QT interval is not prolonged more than about 50 ms. In one embodiment, the QT interval is not prolonged more than about 40 ms. In one embodiment, the QT interval is not prolonged more than about 30 ms. In one embodiment, the QT interval is not prolonged more than about 20 ms. In one embodiment, the QT interval is not prolonged more than about 10 ms.

In one embodiment, the average serum concentration of noribogaine is from 50 ng/mL to 180 ng/mL, or 60 ng/mL to 180 ng/mL. In one embodiment, the average serum concentration of noribogaine is from 50 ng/mL to 150 ng/mL, or 60 ng/mL to 150 ng/mL. In one embodiment, the average serum concentration of noribogaine is from 50 ng/mL to 100 ng/mL, or 60 ng/mL to 100 ng/mL. In one embodiment, the average serum concentration of noribogaine is from 80 ng/mL to 150 ng/mL. In one embodiment, the average serum concentration of noribogaine is from 80 ng/mL to 100 ng/mL. In some aspects of the invention, a lower serum concentration may be therapeutic for a given condition. In one embodiment, the therapeutic serum concentration is between 1 ng/mL and 10 ng/mL. By way of non-limiting example, the therapeutic serum concentration for treatment of nicotine addiction is believed to be lower than that for addiction to opioids. The ranges above include both extremes as well as any subranges between.

In some embodiments, the initial unit dose of noribogaine, noribogaine derivative, or salt or solvate thereof is from 50 mg to 120 mg. In one embodiment, the initial dose is about 50 mg. In one embodiment, the initial dose is about 55 mg. In one embodiment, the initial dose is about 60 mg. In one embodiment, the initial dose is about 65 mg. In one embodiment, the initial dose is about 70 mg. In one embodiment, the initial dose is about 75 mg. In one embodiment, the initial dose is about 80 mg. In one embodiment, the initial dose is about 85 mg. In one embodiment, the initial dose is about 90 mg. In one embodiment, the initial dose is about 95 mg. In one embodiment, the initial dose is about 100 mg. In one embodiment, the initial dose is about 105 mg. In one embodiment, the initial dose is about 110 mg. In one embodiment, the initial dose is about 115 mg. In one embodiment, the initial dose is about 120 mg.

In some embodiments, the initial unit dose of noribogaine, noribogaine derivative, or salt or solvate thereof is administered as subdoses, such that the aggregate dose achieves the unit dose. In some embodiments, the initial unit dose is administered as subunit doses, which subunit doses are administered serially until the unit dose level is achieved, wherein the aggregate of subunit doses provides the initial unit dose and further provides the therapeutic average serum concentration. In some embodiments, the aggregate dose provides a therapeutic serum concentration of 80 ng/mL to 150 ng/mL. In some embodiments, the subdoses are administered every 15 minutes to 6 hours until the unit dose is achieved. In some embodiments, the subdoses are administered every 15 minutes, every 30 minutes, every 1 hour, every 2 hours, every 3 hours, every 4 hours, every 5 hours, or every 6 hours until the unit dose is achieved. The ranges above include both extremes as well as any subranges between.

In some embodiments, the one or more additional doses are lower than the initial dose. In one embodiment, the one or more additional doses are from 5 mg to 75 mg. In one embodiment, the one or more additional doses may or may not comprise the same amount of noribogaine, noribogaine derivative, or salt or solvate thereof. In one embodiment, at least one additional dose is about 5 mg. In one embodiment, at least one additional dose is about 10 mg. In one embodiment, at least one additional dose is about 15 mg. In one embodiment, at least one additional dose is about 20 mg. In one embodiment, at least one additional dose is about 25 mg. In one embodiment, at least one additional dose is about 30 mg. In one embodiment, at least one additional dose is about 35 mg. In one embodiment, at least one additional dose is about 40 mg. In one embodiment, at least one additional dose is about 45 mg. In one embodiment, at least one additional dose is about 50 mg. In one embodiment, at least one additional dose is about 55 mg. In one embodiment, at least one additional dose is about 60 mg. In one embodiment, at least one additional dose is about 65 mg. In one embodiment, at least one additional dose is about 70 mg. In one embodiment, at least one additional dose is about 75 mg. The ranges above include both extremes as well as any subranges between.

In one embodiment, the one or more additional doses are administered periodically. In one embodiment, the one or more additional doses are administered every 4 hours to every 48 hours. In a preferred embodiment, the one or more additional doses are administered every 6 hours to every 24 hours. In one embodiment, the one or more additional doses are administered every 4 hours. In one embodiment, the one or more additional doses are administered every 6 hours. In one embodiment, the one or more additional doses are administered every 8 hours. In one embodiment, the one or more additional doses are administered every 10 hours. In one embodiment, the one or more additional doses are administered every 12 hours. In one embodiment, the one or more additional doses are administered every 18 hours. In one embodiment, the one or more additional doses are administered every 24 hours. In one embodiment, the one or more additional doses are administered every 36 hours. In one embodiment, the one or more additional doses are administered every 48 hours. The ranges above include both extremes as well as any subranges between.

In some embodiments, the therapeutic dose of noribogaine, noribogaine derivative, or salt or solvate thereof is a tapered dosing over a period of time, during which the patient is detoxified, for example, without suffering significant acute withdrawal symptoms. Without being bound by theory, it is believed that tapering will allow the full therapeutic effect of noribogaine with less prolongation of the QT interval. Tapering involves administration of one or more subsequently lower doses of noribogaine over time. For example, in some embodiments, the first tapered dose is 50% to 95% of the initial or at least one additional dose. In some embodiments, the second tapered dose is 40% to 90% of the initial or at least one additional dose. In some embodiments, the third tapered dose is 30% to 85% of the initial or at least one additional dose. In some embodiments, the fourth tapered dose is 20% to 80% of the initial or at least one additional dose. In some embodiments, the fifth tapered dose is 10% to 75% of the initial or at least one additional dose.

In some embodiments, the first tapered dose is given after the first dose of noribogaine. In some embodiments, the first tapered dose is given after the second, third, or a subsequent dose of noribogaine. The first tapered dose may be administered at any time after the previous dose of noribogaine. The first tapered dose can be given once, for example, followed by subsequent further tapered doses, or it can be given multiple times with or without subsequent, further tapered doses (e.g., second, third, fourth, etc. tapered doses), which likewise can be given once or over multiple administrations, for example. In some embodiments, the first tapered dose is administered one hour, 6 hours, 12 hours, 18 hours, 24 hours, 36 hours, 48 hours, or more after the previous dose of noribogaine. Similarly, second, third, fourth, etc. tapered doses, if given, can be given one hour, 6 hours, 12 hours, 18 hours, 24 hours, 36 hours, 48 hours, or more after the previous dose of noribogaine.

In some embodiments, the dose is tapered starting 12 to 96 hours after the initial dose. In some embodiments, the dose is tapered starting 12 hours after the initial dose. In some embodiments, the dose is tapered starting 18 hours after the initial dose. In some embodiments, the dose is tapered starting 24 hours after the initial dose. In some embodiments, the dose is tapered starting 30 hours after the initial dose. In some embodiments, the dose is tapered starting 36 hours after the initial dose. In some embodiments, the dose is tapered starting 42 hours after the initial dose. In some embodiments, the dose is tapered starting 48 hours after the initial dose. In some embodiments, the dose is tapered starting 54 hours after the initial dose. In some embodiments, the dose is tapered starting 60 hours after the initial dose. In some embodiments, the dose is tapered starting 66 hours after the initial dose. In some embodiments, the dose is tapered starting 72 hours after the initial dose. In some embodiments, the dose is tapered starting 78 hours after the initial dose. In some embodiments, the dose is tapered starting 84 hours after the initial dose. In some embodiments, the dose is tapered starting 90 hours after the initial dose. In some embodiments, the dose is tapered starting 96 hours after the initial dose.

In some embodiments, one tapered dose is given to achieve the desired lower therapeutic dose. In some embodiments, two tapered doses are given to achieve the desired lower therapeutic dose. In some embodiments, three tapered doses are given to achieve the desired lower therapeutic dose. In some embodiments, four or more tapered doses are given to achieve the desired lower therapeutic dose. Determination of the tapered doses, number of tapered doses, and the like can be readily made a qualified clinician.

In some embodiments, at least one additional dose is administered 4 hours to 24 hours after the initial unit dose. In some embodiments, the additional doses are administered 4 hours to 24 hours after the previous dose. In some embodiments, the doses are administered every 4 hours to 24 hours. In some embodiments, the doses are administered as needed. The dosage and frequency of the administration depends on the route of administration, dosage, age and body weight of the patient, condition of the patient, without limitation. Determination of dosage and frequency suitable for the present technology can be readily made a qualified clinician.

In some embodiments, the doses are administered at varying time points. That is, each dose need not be administered at the same interval as the previous dose. In some embodiments, the additional doses are administered more frequently at the beginning of treatment, and less frequently after a certain period of time. For example and without limitation, withdrawal symptoms are the most severe in the first 72 hours after the last dose of the drug of addiction. Noribogaine, noribogaine derivative, or a pharmaceutically acceptable solvate or salt thereof may be administered, for example, every 4 hours to 12 hours for the first 72 hours, and less frequently (e.g., 12 hours to 24 hours) thereafter.

Noribogaine, noribogaine derivative, or a pharmaceutically acceptable solvate or salt thereof, suitable for administration in accordance with the methods provide herein, can be suitable for a variety of delivery modes including, without limitation, oral and transdermal delivery. Compositions suitable for internal, pulmonary, rectal, nasal, vaginal, lingual, intravenous, intra-arterial, intramuscular, intraperitoneal, intracutaneous and subcutaneous routes may also be used. Possible dosage forms include tablets, capsules, pills, powders, aerosols, suppositories, parenterals, and oral liquids, including suspensions, solutions and emulsions. Sustained- (controlled- or time-) release dosage forms may also be used. All dosage forms may be prepared using methods that are standard in the art (see e.g., Remington's Pharmaceutical Sciences, 16th ed., A. Oslo editor, Easton Pa. 1980).

In a preferred embodiment, noribogaine, noribogaine derivative, or a pharmaceutically acceptable salt or solvate thereof is administered orally, which may conveniently be provided in tablet, caplet, sublingual, liquid or capsule form. In certain embodiments, the noribogaine is provided as noribogaine HCl, with dosages reported as the amount of free base noribogaine. In some embodiments, the noribogaine HCl is provided in hard gelatin capsules containing only noribogaine HCl with no excipients.

In some embodiments, the noribogaine, noribogaine derivative, or a pharmaceutically acceptable salt or solvate thereof is administered for an indefinite period of time (e.g., for several months or several years, up to the lifetime of the patient).

In some embodiments, the patient undergoes long-term (e.g., one year or longer) treatment with maintenance doses of noribogaine, noribogaine derivative, or salt or solvate thereof. In some embodiments, the patient is first treated for acute symptoms of the condition with therapeutic doses of noribogaine as described above, and then the amount of noribogaine is reduced to maintenance levels, for example after acute symptoms would be expected to have subsided. This is particularly relevant to treating drug addiction, as acute withdrawal symptoms generally are the most pronounced in the first 48 to 72 hours after cessation of the drug of addiction, although acute withdrawal may last as long as a week or more.

In some embodiments, the patient is administered a high (therapeutic) dose of noribogaine, noribogaine derivative, or pharmaceutically acceptable salt or solvate thereof for a period of time to ameliorate the most significant withdraw symptoms, and then is administered a lower (maintenance) dose to prevent relapse to opioid or opioid-like drug use. In some embodiments, the patient is administered a therapeutic dose of noribogaine, noribogaine derivative, or pharmaceutically acceptable salt or solvate thereof for a period of time to ameliorate the most significant withdraw symptoms, and then is administered a decreasing (tapered) amount of noribogaine, noribogaine derivative, or pharmaceutically acceptable salt or solvate thereof over time until the maintenance dose is reached.

In some embodiments, the maintenance dose of noribogaine, noribogaine derivative, or pharmaceutically acceptable salt or solvate thereof is 70% of the therapeutic dose. In some embodiments, the maintenance dose is 60% of the therapeutic dose. In some embodiments, the maintenance dose is 50% of the therapeutic dose. In some embodiments, the maintenance dose is 40% of the therapeutic dose. In some embodiments, the maintenance dose is 30% of the therapeutic dose. In some embodiments, the maintenance dose is 20% of the therapeutic dose. In some embodiments, the maintenance dose is 10% of the therapeutic dose.

In some embodiments, the maintenance average serum level of noribogaine is 70% of the therapeutic average serum level of noribogaine. In some embodiments, the maintenance average serum level of noribogaine is 60% of the therapeutic average serum level of noribogaine. In some embodiments, the maintenance average serum level of noribogaine is 50% of the therapeutic average serum level of noribogaine. In some embodiments, the maintenance average serum level of noribogaine is 40% of the therapeutic average serum level of noribogaine. In some embodiments, the maintenance average serum level of noribogaine is 30% of the therapeutic average serum level of noribogaine. In some embodiments, the maintenance average serum level of noribogaine is 20% of the therapeutic average serum level of noribogaine. In some embodiments, the maintenance average serum level of noribogaine is 10% of the therapeutic average serum level of noribogaine.

### Patient Pre-Screening and Monitoring

Pre-screening of patients before treatment with noribogaine and/or monitoring of patients during noribogaine treatment may be required to ensure that QT interval is not prolonged beyond a certain value. For example, QT interval greater than 500 ms can be considered dangerous for individual patients. Pre-screening and/or monitoring may be necessary at high levels of noribogaine treatment.

In one embodiment, this invention relates to pre-screening a patient to determine if the patient is at risk for prolongation of the QT interval beyond a safe level. In one embodiment, a patient at risk for prolongation of the QT interval beyond a safe level is not administered noribogaine. In one embodiment, a patient at risk for prolongation of the QT interval beyond a safe level is administered noribogaine at a limited dosage.

As it relates to pre-screening or pre-selection of patients, patients may be selected based on any criteria as determined by the skilled clinician. Such criteria may include, by way of non-limiting example, pre-treatment QT interval, pre-existing cardiac conditions, risk of cardiac conditions, age, sex, general health, and the like. The following are examples of selection criteria for disallowing noribogaine treatment or restricting dose of noribogaine administered to the patient: high QT interval before treatment (e.g., such that there is a risk of the patient's QT interval exceeding 500 ms during treatment); congenital long QT syndrome; bradycardia; hypokalemia or hypomagnesemia; recent acute myocardial infarction; uncompensated heart failure; and taking other drugs that increase QT interval. In some embodiments, the methods can include selecting and/or administering/providing noribogaine to a patient that lacks one more of such criteria.

In one embodiment, this invention relates to monitoring a patient who is administered a therapeutic dose of noribogaine. In one embodiment, the dose of noribogaine is reduced if the patient has serious adverse side effects. In one embodiment, the noribogaine treatment is discontinued if the patient has serious adverse side effects. In one embodiment, the adverse side effect is a QT interval that is prolonged beyond a safe level. The determination of a safe level of prolongation is within the skill of a qualified clinician.

### Kit of Parts

One aspect of this invention is directed to a kit of parts for the treatment a condition in a patient which is treatable with noribogaine, noribogaine derivative, or pharmaceutically acceptable salt or solvate thereof, wherein the kit comprises a composition comprising noribogaine, noribogaine derivative, or salt or solvate thereof and a means for administering the composition to a patient in need thereof. The means for administration to a patient can include, for example, any one or combination of a pharmaceutically acceptable formulation comprising noribogaine, or a noribogaine derivative, or a pharmaceutically acceptable salt or solvate thereof (e.g., a pill, transdermal patch, injectable, and the like, without limitation) and optionally a means for dispensing and/or administering the formulation (e.g., a syringe, a needle, an IV bag comprising the composition, a vial comprising the composition, an inhaler comprising the composition, etc, without limitation). In one embodiment, the kit of parts further comprises instructions for dosing and/or administration of the composition.

In some aspects, the invention is directed to a kit of parts for administration of noribogaine, the kit comprising multiple delivery vehicles, wherein each delivery vehicle contains a discrete amount of noribogaine and further wherein each delivery vehicle is identified by the amount of noribogaine provided therein; and optionally further comprising a dosing treatment schedule in a readable medium. In some embodiments, the dosing treatment schedule includes the amount of noribogaine required to achieve each average serum level. In some embodiments, the kit of parts includes a dosing treatment schedule that provides an attending clinician the ability to select a dosing regimen of noribogaine based on the sex of the patient, mass of the patient, and the serum level that the clinician desires to achieve. In some embodiments, the dosing treatment schedule further provides information corresponding to the volume of blood in a patient based upon weight (or mass) and sex of the patient. In an embodiment, the storage medium can include an accompanying pamphlet or similar written information that accompanies the unit dose form in the kit. In an embodiment, the storage medium can include electronic, optical, or other data storage, such as a non-volatile memory, for example, to store a digitally-encoded machine-readable representation of such information.

The term “delivery vehicle” as used herein refers to any formulation that can be used for administration of noribogaine to a patient. Non-limiting, exemplary delivery vehicles include caplets, pills, capsules, tablets, powder, liquid, or any other form by which the drug can be administered. Delivery vehicles may be intended for administration by oral, inhaled, injected, or any other means.

The term “readable medium” as used herein refers to a representation of data that can be read, for example, by a human or by a machine. Non-limiting examples of human-readable formats include pamphlets, inserts, or other written forms. Non-limiting examples of machine-readable formats include any mechanism that provides (i.e., stores and/or transmits) information in a form readable by a machine (e.g., a computer, tablet, and/or smartphone). For example, a machine-readable medium includes read-only memory (ROM); random access memory (RAM); magnetic disk storage media; optical storage media; and flash memory devices. In one embodiment, the machine-readable medium is a CD-ROM. In one embodiment, the machine-readable medium is a USB drive. In one embodiment, the machine-readable medium is a Quick Response Code (QR Code) or other matrix barcode.

In some aspects, the machine-readable medium comprises software that contains information regarding dosing schedules for the unit dose form of noribogaine and optionally other drug information. In some embodiments, the software may be interactive, such that the attending clinician or other medical professional can enter patient information. In a non-limiting example, the medical professional may enter the weight and sex of the patient to be treated, and the software program provides a recommended dosing regimen based on the information entered. The amount and timing of noribogaine recommended to be delivered will be within the dosages that result in the serum concentrations as provided herein.

In some embodiments, the kit of parts comprises multiple delivery vehicles in a variety of dosing options. For example, the kit of parts may comprise pills or tablets in multiple dosages, such as 120 mg, 90 mg, 60 mg, 30 mg, 20 mg, 10 mg, and/or 5 mg of noribogaine per pill. Each pill is labeled such that the medical professional and/or patient can easily distinguish different dosages. Labeling may be based on printing or embossing on the pill, shape of the pill, color of pill, the location of the pill in a separate, labeled compartment within the kit, and/or any other distinguishing features of the pill. In some embodiments, all of the delivery vehicles within a kit are intended for one patient. In some embodiments, the delivery vehicles within a kit are intended for multiple patients.

One aspect of this invention is directed to a kit of parts for the treatment, prevention, or attenuation of a disease or disorder or symptoms of a disease or disorder described herein, wherein the kit comprises a unit dose form of noribogaine, noribogaine derivative, or salt or solvate thereof. The unit dose form provides a patient with an average serum level of noribogaine of from about 50 ng/mL to about 180 ng/mL or about 60 ng/mL to about 180 ng/mL. The unit dose form provides a patient with an average serum level of noribogaine of from about 50 ng/mL to about 800 ng/mL or about 60 ng/mL to about 800 ng/mL. In one embodiment, the unit dose form provides a patient with an average serum level of noribogaine of from about 50 ng/mL to about 400 ng/mL or about 60 ng/mL to about 400 ng/mL. In one embodiment, the unit dose form provides a patient with an average serum level of noribogaine of from 80 ng/mL to 100 ng/mL.

In some embodiments, the unit dose of noribogaine, noribogaine derivative, or pharmaceutically acceptable salt or solvate thereof is from 20 mg to 120 mg. In one embodiment, the unit dose is 20 mg. In one embodiment, the unit dose is 30 mg. In one embodiment, the unit dose is 40 mg. In one embodiment, the unit dose is 50 mg. In one embodiment, the unit dose is 60 mg. In one embodiment, the unit dose is 70 mg. In one embodiment, the unit dose is 80 mg. In one embodiment, the unit dose is 90 mg. In one embodiment, the unit dose is 100 mg. In one embodiment, the unit dose is 110 mg. In one embodiment, the unit dose is 120 mg.

In some embodiments, the unit dose form comprises one or multiple dosages to be administered periodically, such as once, twice, three times, four times or five times daily with noribogaine or its prodrug. In some embodiments, the administration is once daily, or once every second day, once every third day, three times a week, twice a week, or once a week. The dosage and frequency of the administration depends on criteria including the route of administration, content of composition, age and body weight of the patient, condition of the patient, sex of the patient, without limitation, as well as by the severity of the addiction. Determination of the unit dose form providing a dosage and frequency suitable for a given patient can readily be made by a qualified clinician.

In some embodiments, the initial unit dose and one or more additional doses of noribogaine, noribogaine derivative, or salt or solvate thereof are provided as one or multiple dosages to be administered periodically, such as once, twice, three times, four times or five times daily with noribogaine or its prodrug. In some embodiments, the administration is once daily, or once every second day, once every third day, three times a week, twice a week, or once a week. The dosage and frequency of the administration depends on criteria including the route of administration, content of composition, age and body weight of the patient, condition of the patient, sex of the patient, without limitation, as well as by the severity of the addiction. Determination of the unit dose form providing a dosage and frequency suitable for a given patient can readily be made by a qualified clinician.

In one aspect, provided herein is a kit of parts comprising two or more doses of noribogaine, noribogaine derivative, or pharmaceutically acceptable salt or solvate thereof, wherein the two or more doses comprise an amount of noribogaine, noribogaine derivative, or pharmaceutically acceptable salt or solvate thereof that is sufficient to maintain a serum concentration of 50 ng/mL to 180 ng/mL when administered to a patient.

In one embodiment, one dose comprises an initial dose of noribogaine, noribogaine derivative, or pharmaceutically acceptable salt or solvate thereof, said initial dose being sufficient to achieve a therapeutic serum concentration when administered to a patient; and at least one additional dose, said additional dose sufficient to maintain a therapeutic serum concentration when administered to a patient, wherein the therapeutic serum concentration is between 50 ng/mL and 180 ng/mL In another embodiment, the initial dose is from 75 mg to 120 mg. In another embodiment, the at least one additional dose is from 5 mg to 25 mg.

These dose ranges may be achieved by transdermal, oral, or parenteral administration of noribogaine, noribogaine derivative, or a pharmaceutically acceptable salt or solvate thereof in unit dose form. Such unit dose form may conveniently be provided in transdermal patch, tablet, caplet, liquid or capsule form. In certain embodiments, the noribogaine is provided as noribogaine HCl, with dosages reported as the amount of free base noribogaine. In some embodiments, the noribogaine HCl is provided in hard gelatin capsules containing only noribogaine HCl with no excipients. In some embodiments, noribogaine is provided in saline for intravenous administration.

### Formulations

This invention further relates to pharmaceutically acceptable formulations comprising a unit dose of noribogaine, noribogaine derivative, or pharmaceutically acceptable salt or solvate thereof, wherein the amount of noribogaine is sufficient to provide and/or maintain an average serum concentration of 50 ng/mL to 180 ng/mL when administered to a patient. In a preferred embodiment, the amount of noribogaine is sufficient to provide and/or maintain an average serum concentration of 80 ng/mL to 100 ng/mL when administered to a patient.

In some embodiments, the unit dose of noribogaine is administered in one or more dosings.

In one embodiment, the amount of noribogaine is sufficient to provide an average serum concentration of noribogaine from 50 ng/mL to 180 ng/mL, or 60 ng/mL to 180 ng/mL. In one embodiment, the amount of noribogaine is sufficient to provide an average serum concentration of noribogaine from 50 ng/mL to 150 ng/mL, or 60 ng/mL to 150 ng/mL. In one embodiment, the amount of noribogaine is sufficient to provide an average serum concentration of noribogaine from 50 ng/mL to 120 ng/mL, or 60 ng/mL to 120 ng/mL. In one embodiment, the amount of noribogaine is sufficient to provide an average serum concentration of noribogaine from 50 ng/mL to 100 ng/mL, or 60 ng/mL to 100 ng/mL. In one embodiment, the amount of noribogaine is sufficient to provide an average serum concentration of noribogaine from 80 ng/mL to 100 ng/mL. The ranges include both extremes as well as any subranges between.

In some embodiments, the initial unit dose of noribogaine, noribogaine derivative, or pharmaceutically acceptable salt or solvate thereof is from 50 mg to 120 mg. In one embodiment, the unit dose is 50 mg. In one embodiment, the unit dose is 55 mg. In one embodiment, the unit dose is 60 mg. In one embodiment, the unit dose is 65 mg. In one embodiment, the unit dose is 70 mg. In one embodiment, the unit dose is 75 mg. In one embodiment, the unit dose is 80 mg. In one embodiment, the unit dose is 85 mg. In one embodiment, the unit dose is 90 mg. In one embodiment, the unit dose is 95 mg. In one embodiment, the unit dose is 100 mg. In one embodiment, the unit dose is 105 mg. In one embodiment, the unit dose is 110 mg. In one embodiment, the unit dose is 115 mg. In one embodiment, the unit dose is 120 mg.

In some embodiments, the at least one additional dose of noribogaine, noribogaine derivative, or pharmaceutically acceptable salt or solvate thereof is from 5 mg to 75 mg. In one embodiment, the unit dose is 5 mg. In one embodiment, the unit dose is 10 mg. In one embodiment, the unit dose is 15 mg. In one embodiment, the unit dose is 20 mg. In one embodiment, the unit dose is 25 mg. In one embodiment, the unit dose is 30 mg. In one embodiment, the unit dose is 35 mg. In one embodiment, the unit dose is 40 mg. In one embodiment, the unit dose is 45 mg. In one embodiment, the unit dose is 50 mg. In one embodiment, the unit dose is 55 mg. In one embodiment, the unit dose is 60 mg. In one embodiment, the unit dose is 65 mg. In one embodiment, the unit dose is 70 mg. In one embodiment, the unit dose is 75 mg.

In some embodiments, the formulation comprises a delivery vehicle, as described above. In one embodiment, the delivery vehicle comprises 5 mg to 120 mg noribogaine, noribogaine derivative, or pharmaceutically acceptable salt or solvate thereof.

In some embodiments, the formulation is designed for periodic administration, such as once, twice, three times, four times or five times daily with noribogaine, noribogaine derivative, or a pharmaceutically acceptable salt or solvate thereof. In some embodiments, the administration is once daily, or once every second day, once every third day, three times a week, twice a week, or once a week. The dosage and frequency of the administration depends on the route of administration, content of composition, age and body weight of the patient, condition of the patient, without limitation. Determination of dosage and frequency suitable for the present technology can be readily made a qualified clinician.

In some embodiments, the formulation is a controlled release formulation. The term “controlled release formulation” includes sustained release and time-release formulations. Controlled release formulations are well-known in the art. These include excipients that allow for sustained, periodic, pulse, or delayed release of the drug. Controlled release formulations include, without limitation, embedding of the drug into a matrix; enteric coatings; microencapsulation; gels and hydrogels; implants; transdermal patches; and any other formulation that allows for controlled release of a drug.

In some embodiments, the formulation designed for administration in accordance with the methods provide herein can be suitable for a variety of delivery modes including, without limitation, oral, transdermal, sublingual, buccal, intrapulmonary or intranasal delivery. Formulations suitable for internal, pulmonary, rectal, nasal, vaginal, lingual, intravenous, intra-arterial, intramuscular, intraperitoneal, intracutaneous and subcutaneous routes may also be used. Possible formulations include tablets, capsules, pills, powders, aerosols, suppositories, parenterals, and oral liquids, including suspensions, solutions and emulsions. Sustained release dosage forms may also be used. All formulations may be prepared using methods that are standard in the art (see e.g., Remington's Pharmaceutical Sciences, 16th ed., A. Oslo editor, Easton Pa. 1980).

In a preferred embodiment, the formulation is designed for oral administration, which may conveniently be provided in tablet, caplet, sublingual, liquid or capsule form. In certain embodiments, the noribogaine is provided as noribogaine HCl, with dosages reported as the amount of free base noribogaine. In some embodiments, the noribogaine HCl is provided in hard gelatin capsules containing only noribogaine HCl with no excipients.

Noribogaine or a noribogaine derivative can also be used in conjunction with any of the vehicles and excipients commonly employed in pharmaceutical preparations, e.g., talc, gum Arabic, lactose, starch, magnesium stearate, cocoa butter, aqueous or non-aqueous solvents, oils, paraffin derivatives, glycols, etc. Coloring and flavoring agents may also be added to preparations, particularly to those for oral administration. Solutions can be prepared using water or physiologically compatible organic solvents such as ethanol, 1,2-propylene glycol, polyglycols, dimethylsulfoxide, fatty alcohols, triglycerides, partial esters of glycerine and the like. Parenteral compositions containing noribogaine may be prepared using conventional techniques that may include sterile isotonic saline, water, 1,3-butanediol, ethanol, 1,2-propylene glycol, polyglycols mixed with water, Ringer's solution, etc.

The compositions utilized herein may be formulated for aerosol administration, particularly to the respiratory tract and including intrapulmonary or intranasal administration. The compound will generally have a small particle size, for example of the order of 5 microns or less. Such a particle size may be obtained by means known in the art, for example by micronization. The active ingredient may be provided in a pressurized pack with a suitable propellant such as a chlorofluorocarbon (CFC), (for example, dichlorodifluoromethane, trichlorofluoromethane, or dichlorotetrafluoroethane), carbon dioxide or other suitable gases. The aerosol may conveniently also contain a surfactant such as lecithin. The dose of drug may be controlled by a metered valve. Alternatively, the active ingredients may be provided in the form of a dry powder, for example a powder mix of the compound in a suitable powder base such as lactose, starch, starch derivatives such as hydroxypropylmethyl cellulose and polyvinylpyrrolidine. In some embodiments, the powder carrier will form a gel in the nasal cavity. The powder composition may be presented in unit dose form, for example in capsules or cartridges, gelatin or blister packs, from which the powder may be administered by means of an inhaler.

The compositions utilized herein may be formulated for sublingual administration, for example as sublingual tablets. Sublingual tablets are designed to dissolve very rapidly. The formulations of these tablets contain, in addition to the drug, a limited number of soluble excipients, usually lactose and powdered sucrose, but sometimes dextrose and mannitol.

It has been discovered that noribogaine has a bitter taste to at least some patients. Accordingly, compositions for oral use (including sublingual, inhaled, and other oral formulations) may be formulated to utilize taste-masking technologies. A number of ways to mask the taste of bitter drugs are known in the art, including addition of sugars, flavors, sweeteners, or coatings; use of lipoproteins, vesicles, and/or liposomes; granulation; microencapsulation; numbing of taste buds; multiple emulsion; modification of viscosity; prodrug or salt formation; inclusion or molecular complexes; ion exchange resins; and solid dispersion. Any method of masking the bitterness of the compound of the invention may be used.

### EXAMPLES

The following Examples are intended to further illustrate certain embodiments of the disclosure and are not intended to limit its scope.

### Example 1

### Pharmacokinetics and Pharmacodynamics of Noribogaine in Humans

Thirty-six healthy, drug-free male volunteers, aged between 18-55 years, were enrolled in and completed the study. This was an ascending single-dose, placebo-controlled, randomized double blind, parallel group study. Mean (SD) age was 22.0 (3.3) years, mean (SD) height was 1.82 (0.08) m, and mean (SD) weight was 78.0 (9.2) kg. Twenty-six subjects were Caucasian, 3 were Asian, 1 Maori, 1 Pacific Islander, and 5 Other. The protocol for this study was approved by the Lower South Regional Ethics Committee (LRS/12/06/015), and the study was registered with the Australian New Zealand Clinical Trial Registry (ACTRN12612000821897). All subjects provided signed informed consent prior to enrolment, and were assessed as suitable to participate based on review of medical history, physical examination, safety laboratory tests, vital signs and ECG.

Within each dose level, 6 participants were randomized to receive noribogaine and 3 to receive placebo, based on a computer-generated random code. Dosing began with the lowest noribogaine dose, and subsequent cohorts received the next highest dose after the safety, tolerability, and blinded pharmacokinetics of the completed cohort were reviewed and dose-escalation approved by an independent Data Safety Monitoring Board. Blinded study drug was administered as a capsule with 240 ml of water after an overnight fast of at least 10 hours. Participants did not receive any food until at least 5 hours post-dose. Participants were confined to the study site from 12 hours prior to drug administration, until 72 hours post-dose, and there were subsequent outpatient assessments until 216 hours post-dose.

Blood was obtained for pharmacokinetic assessments pre-dose and then at 0.25, 0.5, 0.75, 1.0, 1.25, 1.5, 2, 2.5, 3, 3.5, 4, 4.5, 5, 5.5, 6, 7, 8, 10, 12, 14, 18, 24, 30, 36, 48, 60, 72, 96, 120, 168 and 216 hours post-dose. Samples were centrifuged and plasma stored at −70° C. until analyzed. Block 24 hour urine collections were obtained following study drug administration for the 30 and 60 mg cohorts. Aliquots were frozen at −20° C. until analyzed.

Pulse oximetry and capnography data were collected continuously using a GE Carescape B650 monitoring system from 2 hours prior to dosing and until six hours after dosing, and thereafter at 12, 24, 48 and 72 hours post-dosing. Additional oximetry data were collected at 120, 168 and 216 hours. Pupillary miosis was assessed by pupillometry. Dark-adapted pupil diameter was measured in triplicate using a Neuroptics PLR-200 pupillometer under standardized light intensity (<5 lux) pre-dose, and at 2, 4, 6, 12, 24, 48, 72, 96, 120, 168 and 216 hours post-dosing.

Plasma noribogaine concentrations were determined in the 3 mg and 10 mg dose groups using a validated, sensitive LCMSMS method. Sample preparation involved double extraction of basified plasma samples with tert-butyl methyl ether, drying the samples under a stream of nitrogen and reconstitution of sample with acetonitrile:B.P. water (5:95, v/v) containing 0.1% (v/v) formic acid. The compounds were separated by a 150×2.0 mm Luna 5 μm C18 column and detected with a triple-quadrupole API 4000 or 5000 mass spectrometer using electrospray ionization in positive mode and multiple reaction monitoring. Noribogaine-d₄ was used as the internal standard. The precursor-product ion transition values for noribogaine were m/z 297.6->122.3, and for the internal standard noribogaine-d₄ m/z 301.1->122.2. Analyst® software was used for data acquisition and processing. The ratio of the peak area of noribogaine to the internal standard noribogaine-d₄ was used for calibration and measurement of the unknown concentration of noribogaine. The lower limit of quantification (LLOQ) was 0.025 ng/ml noribogaine. The calibration curve was between 0.025 and 25.600 ng/ml noribogaine. Mobile phase A was acetonitrile:B.P. water (5:95, v/v) containing 0.1% (v/v) formic acid, and mobile phase B was acetonitrile:B.P. water (95:5, v/v) containing 0.1% (v/v) formic acid. Total run time was 6 minutes. Binary flow: Initial concentration was 8% mobile phase B; hold at 8% mobile phase B for 0.5 minutes and linear rise to 90% mobile phase B over 1.5 minutes; hold at 90% mobile phase B for 1 minute and then drop back to 8% mobile phase B over 0.01 minute. Equilibrate system for 3 minutes. Total run time was 6 minutes. Within- and between-day assay precision was <9%, and within- and between-day assay accuracy was <9%.

Plasma noribogaine concentrations were determined in the 30 mg and 60 mg dose groups using a validated, sensitive LCMSMS method. Sample preparation involved deproteinization of plasma samples with acetonitrile and dilution of sample with 0.1% (v/v) formic acid. The compounds were separated by a 150×2.0 mm Luna 5 μm C18 column and detected with a triple-quadrupole API 4000 or 5000 mass spectrometer using electrospray ionization in positive mode and multiple reaction monitoring. Noribogaine-d₄ was used as the internal standard. The precursor-product ion transition values for noribogaine were m/z 297.6->122.3, and for the internal standard noribogaine-d₄ m/z 301.1->122.2. Analyst® software was used for data acquisition and processing. The ratio of the peak area of noribogaine to the internal standard noribogaine-d₄ was used for calibration and measurement of the unknown concentration of noribogaine. The LLOQ was 0.50 ng/ml noribogaine. The calibration curve was between 0.50 and 256.00 ng/ml noribogaine. Mobile phase was the same as method A, and binary flow was also the same as method A. The within- and between-day assay precision was <9%, and the within- and between-day assay accuracy was <9%.

Plasma noribogaine glucuronide concentrations were determined in the 30 mg and 60 mg dose groups using a validated sensitive LCMSMS method. Sample preparation involved deproteinization of plasma samples with acetonitrile, drying the samples under a stream of nitrogen and reconstitution of sample with acetonitrile:B.P. water (5:95, v/v) containing 0.1% (v/v) formic acid. The compounds were separated by a 150×2.0 mm Luna 5 μm C18 column and detected with a triple-quadrupole API 4000 or 5000 mass spectrometer using electrospray ionization in positive mode and multiple reaction monitoring. Noribogaine-d₄ was used as the internal standard. The precursor-product ion transition values for noribogaine glucuronide were m/z 472.8->297.3, and for the internal standard noribogaine-d₄ m/z 301.1->122.2. Analyst® software was used for data acquisition and processing. The ratio of the peak area of noribogaine glucuronide to the internal standard noribogaine-d₄ was used for calibration and measurement of the unknown concentration of noribogaine glucuronide. The LLOQ was 0.050 ng/ml noribogaine glucuronide. The calibration curve was between 0.050 and 6.400 ng/ml noribogaine glucuronide. Mobile phases was the same as method A. Binary flow: Initial concentration was 6% mobile phase B; hold at 6% mobile phase B for 0.5 minutes and linear rise to 90% mobile phase B over 2 minutes; hold at 90% mobile phase B for 1 minute and then drop back to 6% mobile phase B over 0.01 minute. Equilibrate system for 3.5 minutes. Total run time was 7 minutes. The within- and between-day assay precision was <11%, and the within- and between-day assay accuracy was <10%.

Urine noribogaine and noribogaine glucuronide concentrations were determined in the 30 mg and 60 mg dose groups using a validated sensitive LCMSMS method. Sample preparation involved deproteinization of urine samples with acetonitrile and dilution of the sample with 0.1% (v/v) formic acid. The compounds were separated by a 150×2.0 mm Luna 5 μm C18 column and detected with a triple-quadrupole API 4000 or 5000 mass spectrometer using electrospray ionization in positive mode and multiple reaction monitoring. Noribogaine-d₄ was used as the internal standard. The precursor-product ion transition values for noribogaine were m/z 297.6->122.3, noribogaine glucuronide m/z 472.8->297.3, and for the internal standard noribogaine-d₄ m/z 301.1->122.2. Analyst® software was used for data acquisition and processing. The ratios of the peak area of noribogaine and noribogaine glucuronide to the internal standard noribogaine-d₄ were used for calibration and measurement of the unknown concentration of noribogaine and its glucuronide. Assay LLOQ was 20.0 ng/ml for noribogaine and 2.0 ng/ml for noribogaine glucuronide. The calibration curve was between 20.0 and 5120.0 ng/ml noribogaine, and 2.0 and 512.0 ng/ml noribogaine glucuronide. Mobile phases were as described in method A, and binary flow as in method C. The within- and between-day assay precision was <13%, and within- and between-day assay accuracy was <12%.

Noribogaine and noribogaine glucuronide concentrations above the limit of quantification were used to calculate pharmacokinetic parameters using model-independent methods. The maximum plasma concentration (Cmax) and time to maximum plasma concentration (Tmax) were the observed values. Plasma concentration data in the post-distribution phase of the plasma concentration-time plot were fitted using linear regression to the formula ln C=ln Co−t·Kel, where Co was the zero-time intercept of the extrapolated terminal phase and Kel was the terminal elimination rate constant. The half-life (t_{1/2}) was determined using the formula t_{1/2}2=0.693/Kel. The area under the concentration-time curve (AUC) from time zero to the last determined concentration-time point (tf) in the post distribution phase was calculated using the trapezoidal rule. The area under the curve from the last concentration-time point in the post distribution phase (Ctf) to time infinity was calculated from AUC_{t-∞}=Ctf/Kel. The concentration used for Ctf was the last determined value above the LLOQ at the time point. The total AUC_{0-∞}was obtained by adding AUC_{tf} and AUC_{t-∞}. Noribogaine apparent clearance (CL/F) was determined using the formula CL/F=Dose/AUC_{0-∞}×1000, and apparent volume of distribution (Vd/F) was determined using the formula Vd/F=(CL/F)/Kel. Total urine noribogaine was the sum of both analytes.

Summary statistics (means, standard deviations, and coefficients of variation) were determined for each dose group for safety laboratory test data, ECG and pharmacokinetic parameters, and pharmacodynamic variables. Categorical variables were analysed using counts and percentages. Dose-proportionality of AUC and Cmax was assessed using linear regression. The effect of dose on pharmacodynamic parameter values over time was assessed using two-factor analysis of variance (ANOVA). Pairwise comparisons (with Tukey-Kramer adjustment) between each dose group to the placebo were conducted at each time point using the least squares estimates obtained from the ANOVA, using SAS Proc Mixed (SAS ver 6.0).

### Results

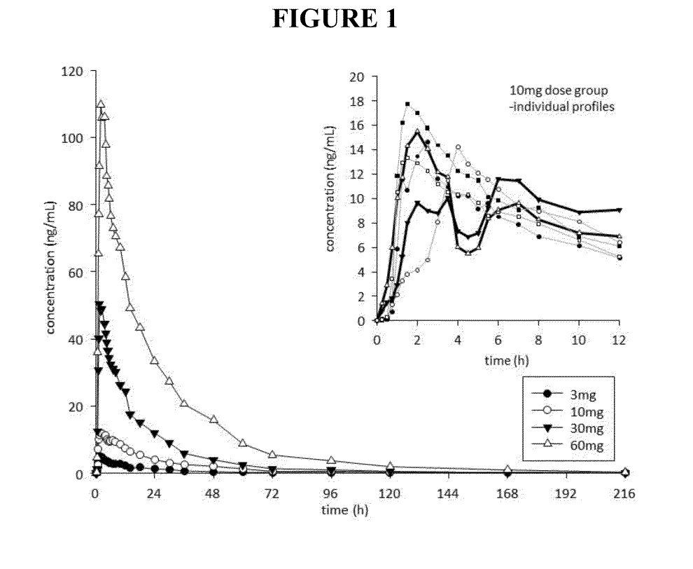

Pharmacokinetics: Mean plasma concentration-time plots of noribogaine are shown in FIG. 1, and mean pharmacokinetic parameters are shown in Table 1.

TABLE 1

| | 3 mg (n = 6) | 10 mg (n = 6) | 30 mg (n = 6) | 60 mg (n = 6) |
|---|---|---|---|---|
| Noribogaine | (mean (SD)) | (mean (SD)) | (mean (SD)) | (mean (SD) |
| AUC_{0-∞} (ng · hr/ml) | 74.2 (13.1) | 254.5 (78.9) | 700.4 (223.3) | 1962.2 (726.5) |
| AUC₀₋₂₁₆ (ng · hr/ml) | 72.2 (13.2) | 251.4 (78.5) | 677.6 (221.1) | 1935.4 (725.4) |
| Cmax (ng/ml) | 5.2 (1.4) | 14.5 (2.1) | 55.9 (14.8) | 116.0 (22.5) |
| Tmax (hr) | 1.9 (0.6) | 2.9 (1.8) | 1.8 (0.6) | 2.4 (0.6) |
| t_{1/2} (hr) | 40.9 (8.7) | 49.2 (11.5) | 27.6 (7.0)) | 29.1 (9.3) |
| Vd/F (L) | 2485.1 (801.5) | 3085.8 (1197.0) | 1850.8 (707.9) | 1416.8 (670.1) |
| CL/F (L/h) | 41.4 (7.0) | 42.3 (12.0) | 46.9 (16.4) | 34.0 (11.4) |

Noribogaine glucuronide

| | | | | |
|---|---|---|---|---|
| AUC_{0-∞} | — | — | 25.8 (9.3) | 67.1 (21.9) |
| (ng · hr/ml) | | | | |
| AUC₀₋₂₁₆ (ng · hr/ml) | — | — | 25.7 (9.1) | 65.0 (21.5) |
| Cmax (ng/ml) | — | — | 1.8 (0.6) | 4.1 (1.2) |
| Tmax (hr) | — | — | 3.0 (0.6) | 3.8 (1.2) |
| t_{1/2} (hr) | — | — | 20.6 (4.9) | 23.1 (3.0) |

Noribogaine was rapidly absorbed, with peak concentrations occurring 2-3 hours after oral dosing. Fluctuations in individual distribution-phase concentration-time profiles may suggest the possibility of enterohepatic recirculation (see highlighted individual 4-8 hour profiles in FIG. 1, insert). Both Cmax and AUC increased linearly with dose (Table 1, upper panel). Mean half-life estimates of 28-50 hours were observed across dose groups for noribogaine. Volume of distribution was extensive (1417-3086 L across dose groups).

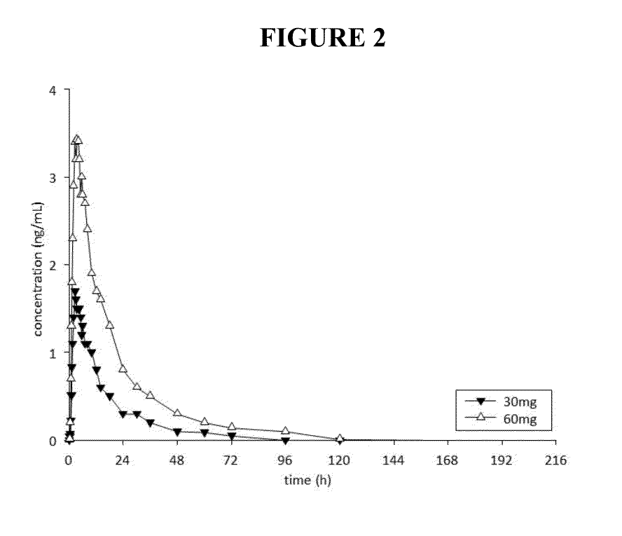

Mean plasma noribogaine glucuronide concentration-time plots for the 30 mg and 60 mg dose group are shown in FIG. 2, and mean pharmacokinetic parameters are shown in Table 1, lower panel. Noribogaine glucuronide was detected in all subjects by 0.75 hours, with peak concentrations occurring 3-4 hours after noribogaine dosing. Mean half-life of 21-23 hours was estimated for plasma noribogaine glucuronide. The proportion of noribogaine glucuronide Cmax and AUC relative to noribogaine was 3-4% for both dose groups. Total urine noribogaine elimination was 1.16 mg and 0.82 mg for the 30 mg and 60 mg dose groups respectively, representing 3.9% and 1.4% of the doses administered.

The subject mean serum levels over time of noribogaine free base from a single dose of 3 mg noribogaine free base under fasting conditions were plotted. The mean Cₘₐₓ of 5.2 ng/ml was observed 1.9 hours after administration, while the mean AUC/24 hr of 3.1 ng/ml was obtained.

The subject mean serum levels over time of noribogaine free base from a single dose of 10 mg noribogaine free base under fasting conditions were plotted. The mean Cₘₐₓ of 14.5 ng/ml was observed 2.9 hours after administration, while the mean AUC/24 hr of 10.6 ng/ml was obtained.

The subject mean serum levels over time of noribogaine free base from a single dose of 30 mg noribogaine free base under fasting conditions were plotted. The mean Cₘₐₓ of 55.9 ng/ml was observed between 1.75 hours after administration, while the mean AUC/24 of 29.2 ng/ml was obtained.

The subject mean serum levels over time of noribogaine free base from a single dose of 60 mg noribogaine free base under fasting conditions were plotted. The mean Cₘₐₓ of 116 ng/ml was observed between 1.75 hours after administration, while the mean AUC/24 ng/ml of 61 was obtained.

The subject mean serum levels over time of noribogaine free base for all 4 cohorts were plotted. The extrapolated dosage of noribogaine free base required to provide a Cₘₐₓ ranging from about 5.2 ng/ml to about 1980 ng/ml and an AUC/24 hr of about 3.1 ng/ml to about 1100 ng/ml was determined.

Pharmacodynamics: There was no evidence of pupillary constriction in subjects dosed with noribogaine. No between-dose group differences in pupil diameter were detected over time. After adjusting for baseline differences, comparison of each dose group with placebo by ANOVA showed no statistically significant differences (p>0.9).

Noribogaine treatment showed no analgesic effect in the cold pressor test. Analgesic effect was assessed based on duration of hand immersion in ice water and on visual analog scale (VAS) pain scores upon hand removal from the water bath. For duration of hand immersion, after adjusting for baseline differences, comparison of each dose group with placebo by ANOVA showed no statistically significant differences (p>0.9). Similarly, for VAS pain scores, after adjusting for baseline differences, comparison of each dose group with placebo by ANOVA showed no statistically significant differences (p=0.17).

### Example 2

### Safety and Tolerability of Noribogaine in Healthy Humans

Safety and tolerability of noribogaine were tested in the group of volunteers from Example 1. Cold pressor testing was conducted in 1° C. water according to the method of Mitchell et al. (J Pain 5:233-237, 2004) pre-dose, 6, 24, 48, 72 and 216 hours post-dosing. Safety evaluations included clinical monitoring, recording of adverse events (AEs), safety laboratory tests, vital signs, ECG telemetry from −2 h to 6 h after dosing, and 12-lead electrocardiograms (ECGs) up to 216 hours post-dosing.

### Results

A total of thirteen adverse events were reported by seven participants (Table 2). Six adverse events were reported by three participants in the placebo group, five adverse events were reported by two subjects in the 3 mg dose group, and one adverse event was reported by single subjects in the 10 mg and 30 mg dose groups, respectively. The most common adverse events were headache (four reports) and epistaxis (two reports). All adverse events were of mild-moderate intensity, and all resolved prior to study completion. There were no changes in vital signs or safety laboratory tests of note. In particular, there were no changes in oximetry or capnography, or changes in respiratory rate. There were no QTcF values>500 msec at any time. One subject dosed with 10 mg noribogaine had a single increase in QTcF of >60 msec at 24 hours post-dosing.

TABLE 2

| Dose | | | |
|---|---|---|---|
| (mg) | Mild | Moderate | Severe |
| Placebo | Blepharitis | Epistaxis | — |
| | Bruising | | |
| | Dry Skin | | |
| | Eye pain, nonspecific | | |
| | Infection at cannula site | | |
| 3 | Back pain | Headache | — |
| | Dizziness | | |
| | Epistaxis | | |
| | Headache | | |
| 10 | Headache | — | — |
| 30 | Headache | — | — |
| 60 | — | — | — |

### Example 3

### Safety. Tolerability, and Efficacy of Noribogaine in Opioid-Addicted Humans

This example is to illustrate that noribogaine can be administered at a therapeutic dosing while maintaining an acceptable QT interval. While the therapy employed is directed to opioid-dependent participants in a randomized, placebo-controlled, double-blind trial, the results show that a therapeutic window can be established for noribogaine.

The efficacy of noribogaine in humans was evaluated in opioid-dependent participants in a randomized, placebo-controlled, double-blind trial. Patients had been receiving methadone treatment as the opioid substitution therapy, but were transferred to morphine treatment prior to noribogaine administration. This was done to avoid negative noribogaine-methadone interactions that are not observed between noribogaine and morphine. See U.S. application Ser. No. 14/214,157, filed Mar. 14, 2014 and Ser. No. 14/346,655, filed Mar. 21, 2014, which are incorporated herein by reference in their entireties.

Three cohorts of nine (9) subjects (6 administered noribogaine and 3 administered placebo in each cohort) were evaluated for tolerability, pharmacokinetics, and efficacy. Cohort 1 received a single dose of 60 mg noribogaine or placebo. Cohort 2 received a single dose of 120 mg noribogaine or placebo. Cohort 3 received a single dose of 180 mg noribogaine or placebo. Treatment was administered 2 hours after last morphine dose and the time to resumption of morphine (opioid substitution treatment, OST) was determined. Few adverse effects of noribogaine were observed in any of the participants, including no hallucinatory effects. Table 3 shows the reported adverse events for each treatment that were not attributable to withdrawal from opioids. Headaches were frequent in the placebo and 60 mg noribogaine treatment groups, but were attenuated in the 120 mg and 180 mg dose groups.

TABLE 3
Treatment Emergent Adverse Events Summary

| System Organ Class | Placebo | 60 mg | 120 mg | 180 mg |
|---|---|---|---|---|
| Preferred Term | (N = 9) | (N = 6) | (N = 6) | (N = 6) |
| Number of Subjects Reporting any AEs | 19:7 (77.8%) | 15:5 (83.3%) | 28:6 (100.0%) | 17:4 (66.7%) |
| Ear and Labyrinth Disorders | 0 | 0 | 2:2 (33.3%) | 0 |
| Tinnitus | 0 | 0 | 2:2 (33.3%) | 0 |
| Eye Disorders | 2:2 (22.2%) | 3:3 (50.0%) | 5:5 (83.3%) | 5:4 (66.7%) |
| Visual Impairment | 2:2 (22.2%) | 2:2 (33.3%) | 5:5 (83.3%) | 5:4 (66.7%) |
| Dry Eye | 0 | 1:1 (16.7%) | 0 | 0 |
| Gastrointestinal Disorders | 3:2 (22.2%) | 2:2 (33.3%) | 7:2 (33.3%) | 4:2 (33.3%) |
| Nausea | 1:1 (11.1%) | 0 | 3:2 (33.3%) | 2:2 (33.3%) |
| Dry Mouth | 0 | 0 | 1:1 (16.7%) | 1:1 (16.7%) |
| Vomiting | 0 | 0 | 2:1 (16.7%) | 1:1 (16.7%) |
| Diarrhoea | 1:1 (11.1%) | 0 | 1:1 (16.7%) | 0 |
| Dyspepsia | 1:1 (11.1%) | 2:2 (33.3%) | 0 | 0 |
| General Disorders and | 4:3 (33.3%) | 0 | 2:2 (33.3%) | 1:1 (16.7%) |
| Administration Site Conditions | | | | |
| Catheter Site Related Reaction | 0 | 0 | 0 | 1:1 (16.7%) |
| Catheter Site Pain | 3:2 (22.2%) | 0 | 2:2 (33.3%) | 0 |
| Malaise | 1:1 (11.1%) | 0 | 0 | 0 |
| Infections and Infestations | 1:1 (11.1%) | 0 | 1:1 (16.7%) | 2:2 (33.3%) |
| Cellulitis | 0 | 0 | 1:1 (16.7%) | 1:1 (16.7%) |
| Urinary Tract Infection | 0 | 0 | 0 | 1:1 (16.7%) |
| Catheter Site Infection | 1:1 (11.1%) | 0 | 0 | 0 |
| Musculoskeletal and Connective Tissue | 1:1 (11.1%) | 2:1 (16.7%) | 0 | 2:2 (33.3%) |
| Disorders | | | | |
| Back Pain | 1:1 (11.1%) | 2:1 (16.7%) | 0 | 1:1 (16.7%) |
| Limb Discomfort | 0 | 0 | 0 | 1:1 (16.7%) |
| Nervous System Disorders | 7:5 (55.6%) | 7:4 (66.7%) | 5:4 (66.7%) | 3:2 (33.3%) |
| Headache | 6:5 (55.6%) | 7:4 (66.7%) | 2:2 (33.3%) | 3:2 (33.3%) |
| Hyperaesthesia | 0 | 0 | 1:1 (16.7%) | 0 |
| Pseudoparalysis | 0 | 0 | 1:1 (16.7%) | 0 |
| Tremor | 0 | 0 | 1:1 (16.7%) | 0 |
| Somnoience | 1:1 (11.1%) | 0 | 0 | 0 |
| Psychiatric Disorders | 1:1 (11.1%) | 1:1 (16.7%) | 0 | 0 |
| Depressed Mood | 0 | 1:1 (16.7%) | 0 | 0 |
| Euphoric Mood | 1:1 (11.1%) | 0 | 0 | 0 |
| Respiratory, Thoracic and Mediastinal | 0 | 0 | 4:2 (33.3%) | 0 |
| Disorders | | | | |
| Epistaxis | 0 | 0 | 2:1 (16.7%) | 0 |
| Oropharyngeal Pain | 0 | 0 | 1:1 (16.7%) | 0 |
| Rhinorrhoea | 0 | 0 | 1:1 (16.7%) | 0 |
| Skin and Subcutaneous Tissue Disorders | 0 | 0 | 2:1 (16.7%) | 0 |
| Skin Discomfort | 0 | 0 | 1:1 (16.7%) | 0 |
| Skin Irritation | 0 | 0 | 1:1 (16.7%) | 0 |

Note:
Within each system organ class, Preferred Terms are presented by descending incidence of descending dosages groups and then the placebo group.
Note:
N = number of subjects in the safety population.

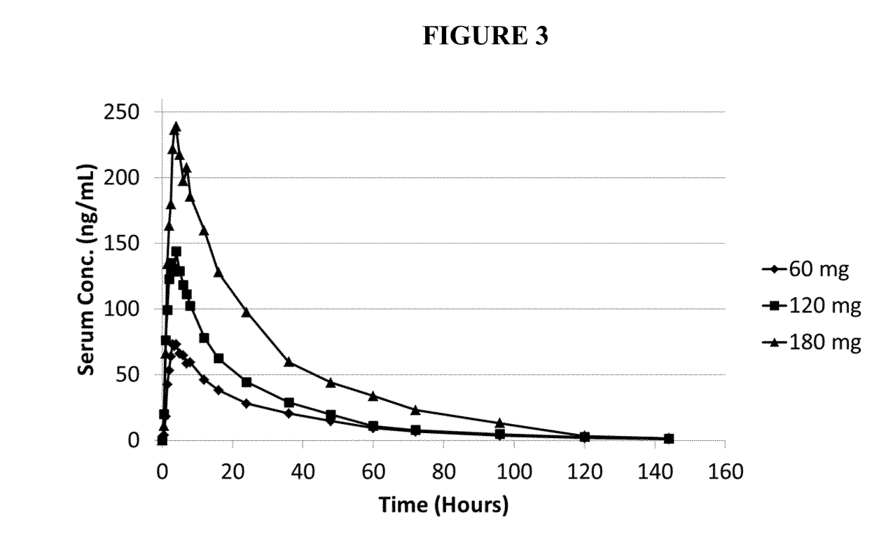

FIG. 3 indicates the average serum noribogaine concentration over time after administration of noribogaine for each cohort (60 mg, diamonds; 120 mg, squares; or 180 mg, triangles). Further results are detailed in U.S. Provisional Patent Application No. 62/023,100, filed Jul. 10, 2014, and titled “METHODS FOR ACUTE AND LONG-TERM TREATMENT OF DRUG ADDICTION,” which is incorporated herein by reference in its entirety.

### Results

Pharmacokinetic results for each cohort are given in Table 4. Maximum serum concentration of noribogaine (Cmax) increased in a dose-dependent manner. Time to Cmax (Tmax) was similar in all three cohorts. Mean half-life of serum noribogaine was similar to that observed in healthy patients.

TABLE 4
Pharmacokinetic results from the Patients in Phase IB Study

| PK | Cohort 1 (60 mg) | Cohort 2 (120 mg) | Cohort 3 (180 mg) |
|---|---|---|---|
| para- | Data (mean ± SD) | Data (mean ± SD) | Data (mean ± SD) |
| meter | [range] | [range] | [range] |
| Cmax | 81.64 ± 23.77 | 172.79 ± 30.73 | 267.88 ± 46.92 |
| (ng/ml) | [41.29 − 113.21] | [138.84 − 229.55] | [204.85 − 338.21] |
| Tmax | 3.59 ± 0.92 | 2.99 ± 1.23 | 4.41 ± 1.80 |
| (hours) | [2.50 − 5.00] | [0.98 − 4.02] | [3.00 − 8.00] |
| AUC_{(0-T)} | 2018.01 ± 613.91 | 3226.38 ± 1544.26 | 6523.28 ± 2909.80 |
| (ng · hr/ | [1094.46 − 2533.44] | [1559.37 − 5638.98] | [3716.69 − 10353.12] |
| ml) | | | |
| AUC_{(0-¥)} | 2060.31 ± 609.39 | 3280.50 ± 1581.43 | 6887.67 ± 3488.91 |
| (ng · hr/ | [1122.29 − 2551.63] | [1595.84 − 5768.52] | [3734.21 − 12280.91] |
| ml) | | | |
| Half-life | 29.32 + 7.28 | 30.45 ± 9.14 | 23.94 ± 5.54 |
| (hrs) | [18.26 − 37.33] | [21.85 − 48.33] | [19.32 − 34.90] |
| Vd/F | 1440.7 ± 854.0 | 2106.43 ± 1644.54 | 1032.19 ± 365.30 |
| | [619.5 − 2772.5] | [824.24 − 5243.78] | [581.18 − 1608.98] |
| Cl/F | 32.14 ± 12.38 | 44.68 ± 21.40 | 31.47 ± 13.12 |
| | [23.51 − 53.46] | [20.80 − 75.20] | [14.66 − 48.20] |

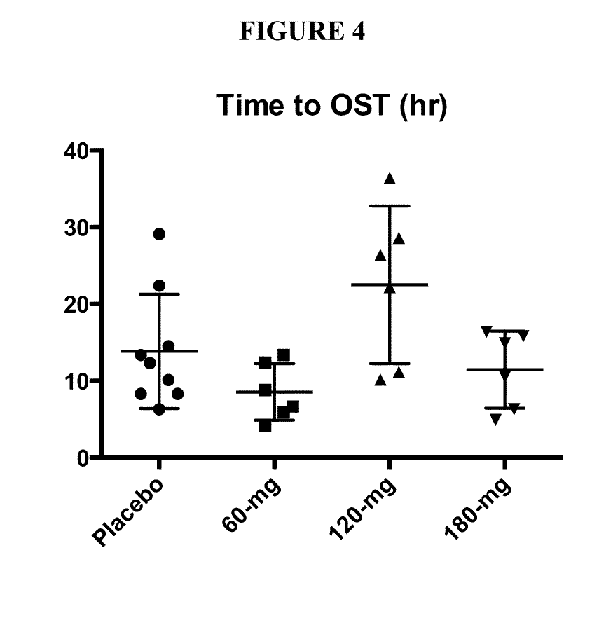

FIG. 4 indicates the time to resumption of morphine (OST) for patients treated with placebo (circles), 60 mg noribogaine (squares), 120 mg noribogaine (triangles), and 180 mg noribogaine (inverted triangles). Patients receiving a single 120 mg dose of noribogaine exhibited an average time to resumption of opioids of greater than 20 hours. Patients receiving a single 180 mg dose of noribogaine exhibited an average time to resumption of opioids similar to that of placebo. This demonstrates that increasing the dose of noribogaine to 180 mg results in a shorter time to resumption of OST than observed in patients receiving 120 mg noribogaine. Time to resumption of OST after treatment with 180 mg was still longer than untreated patients (7 hours, not shown) or those administered 60 mg noribogaine.

Patients were evaluated based on the Clinical Opiate Withdrawal Scale (COWS), Subjective Opiate Withdrawal Scale (SOWS), and Objective Opiate Withdrawal Scale (OOWS) scoring systems over the period of time between administration of noribogaine (or placebo) until resumption of OST. These scales are outlined in Guidelines for the Psychosocially Assisted Pharmacological Treatment of Opioid Dependence, World Health Organization, Geneva (2009), Annex 10, which is incorporated herein by reference in its entirety. The scales measure the intensity of withdrawal symptoms, based on clinical, subjective, and objective indicia.

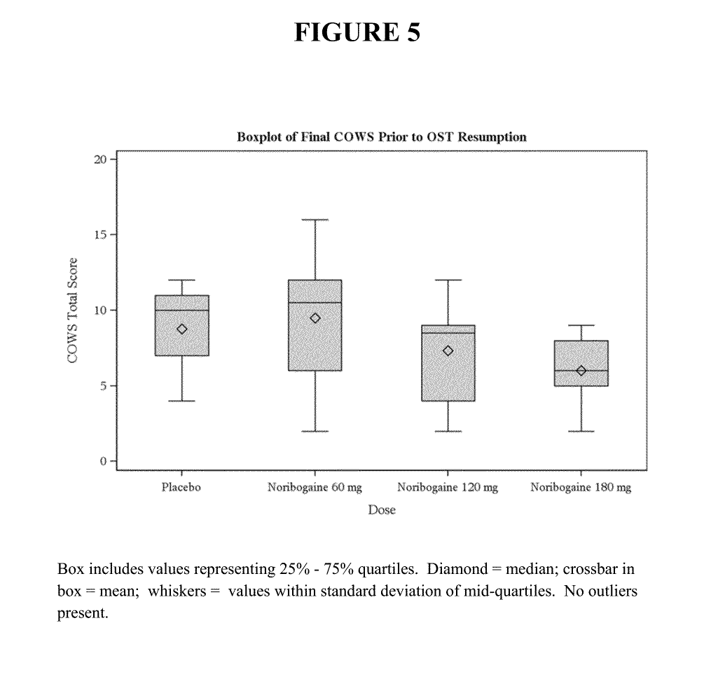

FIG. 5 shows the COWS scores at time of resumption of OST for each cohort. Box includes values representing 25%-75% quartiles. Diamond=median; crossbar in box=mean; whiskers=values within standard deviation of mid-quartiles. No outliers present. The highly variable COWS scores across and within each cohort indicates that patients were resuming opiates without relation to the intensity of withdrawal. This was also reflected in SOWS and OOWS scores at the time of resumption of OST.

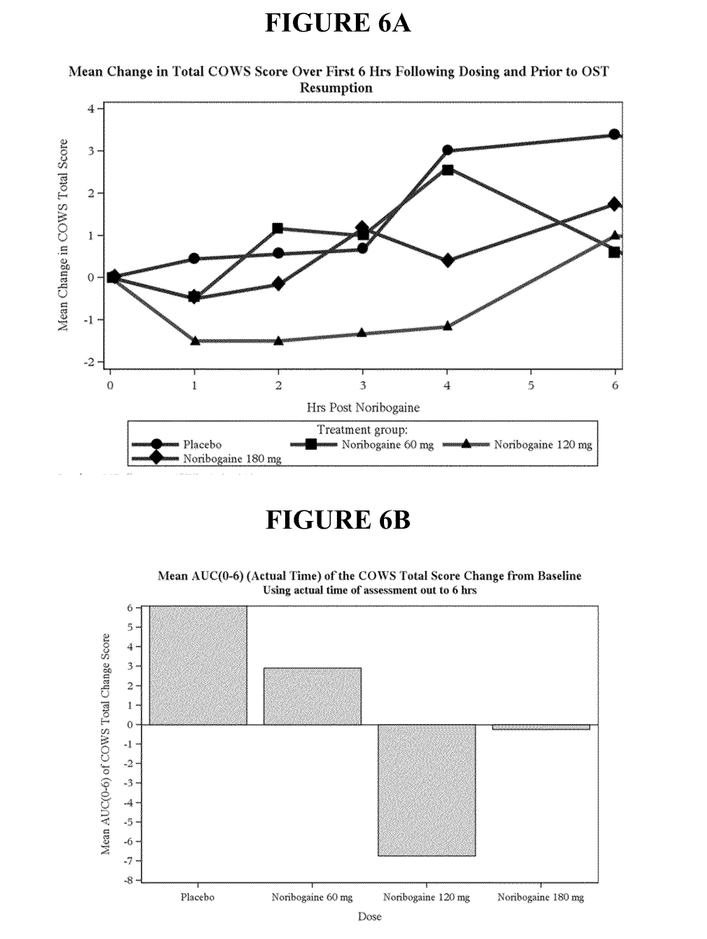

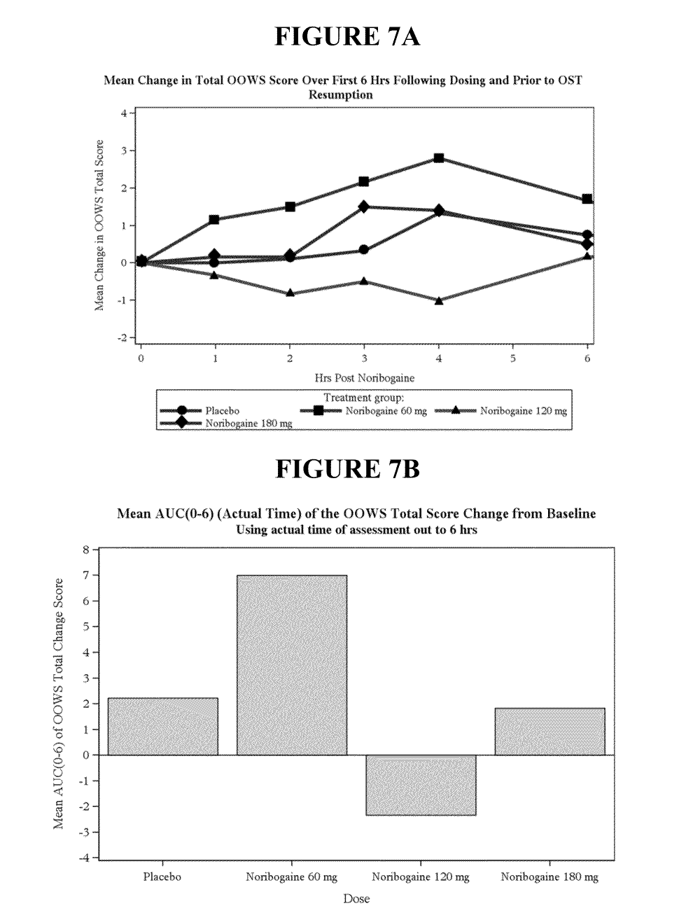

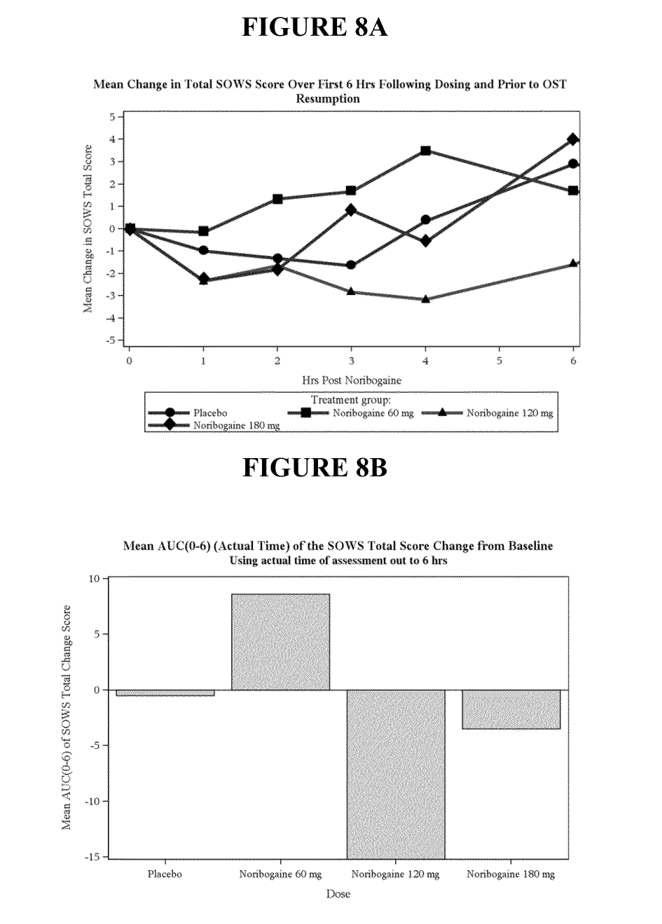

FIG. 6A shows the mean change in total COWS scores over the first six hours following dosing and prior to resumption of OST. FIG. 6B shows the mean AUC(0-6 hours) of the COWS total score change from baseline. FIG. 7A shows the mean change in total OOWS scores over the first six hours following dosing and prior to resumption of OST. FIG. 7B shows the mean AUC(0-6 hours) of the OOWS total score change from baseline. FIG. 8A shows the mean change in total SOWS scores over the first six hours following dosing and prior to resumption of OST. FIG. 8B shows the mean AUC(0-6 hours) of the SOWS total score change from baseline. These data indicate that withdrawal symptoms get worse over time after cessation of OST, and that patients administered placebo experience generally worse withdrawal symptoms over that period. Patients who received 120 mg noribogaine generally experienced fewer withdrawal symptoms than the other patients, regardless of the scale used. Patients administered placebo generally experienced more withdrawal symptoms than patients who were administered noribogaine.

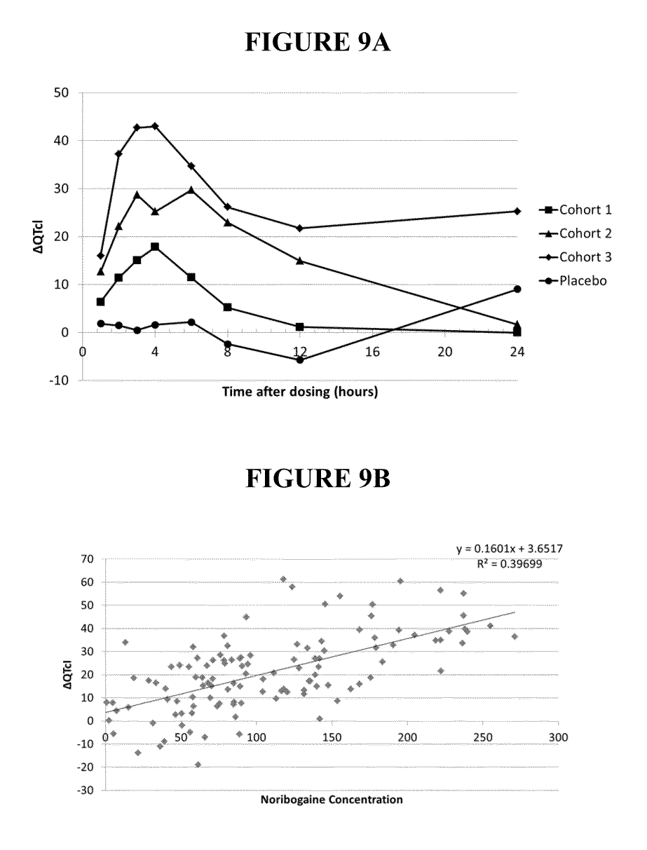

Patients' QT intervals were evaluated at regular time points throughout the study. FIG. 9A shows the average change in QT interval (ΔQTcl, i.e., QT interval prolongation) over the first 24 hours post noribogaine (or placebo) administration. FIG. 9B shows the estimated correlation between noribogaine concentration and change in QT interval. There is a dose-dependent increase in QT interval prolongation that is correlated with the serum concentration of noribogaine.

Based on above data, it is believed that the therapeutic window for a single bolus dose of noribogaine is bound at the lower end by 50 mg and at the upper end by less than 180 mg. In particular, the therapeutic serum concentration in vivo appears to be between about 50 ng/mL and about 180 ng/mL.

### Example 4

### Multiple Dosing of Noribogaine in Humans

The efficacy of multiple dosing of noribogaine in humans is evaluated in opioid-dependent participants in a randomized, placebo-controlled, double-blind trial. Patients are receiving methadone treatment as the opioid substitution therapy, but are transferred to morphine treatment prior to noribogaine administration.

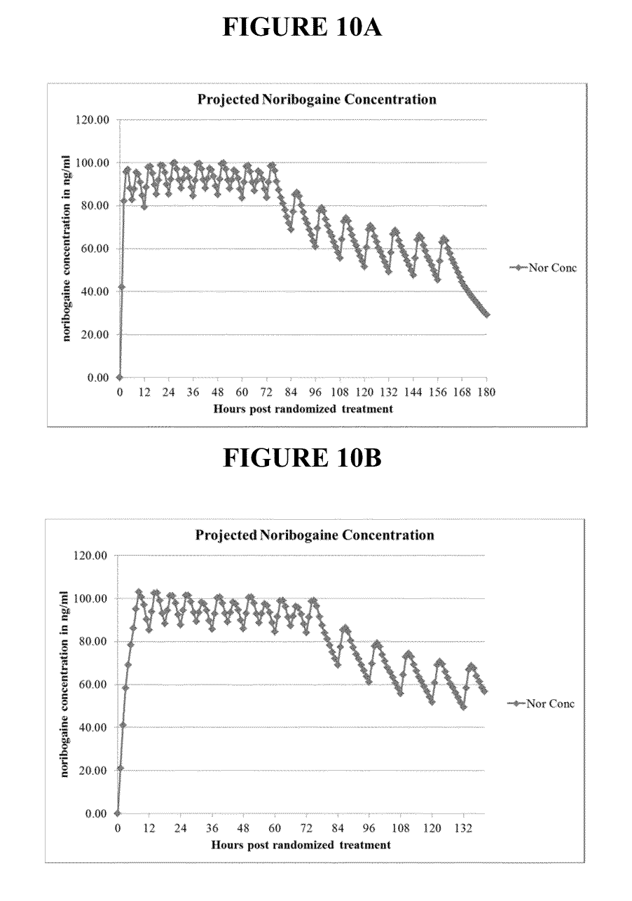

Patients are treated with an initial unit dose of noribogaine of 80 mg, followed by 15 mg to 20 mg of noribogaine every 6 hours to 12 hours, as indicated in Table 5. The noribogaine serum level is indicated in FIG. 10A, based on the data from Example 3.

TABLE 5

| Hour | 0 | 6 | 12 | 18 | 24 | 30 | 36 | 42 | 48 | 54 | 60 | 66 | 72 | 84 | 96 | 108 | 120 | 132 | 144 | 156 |
|---|---|---|---|---|---|---|---|---|---|---|---|---|---|---|---|---|---|---|---|---|
| Dose (mg) | 80 | 20 | 25 | 20 | 20 | 15 | 20 | 15 | 20 | 15 | 20 | 15 | 20 | 20 | 20 | 20 | 20 | 20 | 20 | 20 |

Patients are treated with an initial unit dose of noribogaine of 80 mg, which is administered as subdoses of 40 mg followed by 20 mg at 2 hours and 4 hours after the first dose. The initial unit dose is followed by 15 mg to 20 mg of noribogaine every 6 hours to 12 hours, as indicated in Table 6. The noribogaine serum level is indicated in FIG. 10B, based on the data from Example 3.

TABLE 6

| Hour | 0 | 2 | 4 | 6 | 12 | 18 | 24 | 30 | 36 | 42 | 48 | 54 | 60 | 66 | 72 | 84 | 96 | 108 | 120 | 132 |
|---|---|---|---|---|---|---|---|---|---|---|---|---|---|---|---|---|---|---|---|---|
| Dose (mg) | 40 | 20 | 20 | 20 | 25 | 20 | 20 | 15 | 20 | 15 | 20 | 15 | 20 | 15 | 20 | 20 | 20 | 20 | 20 | 20 |

What is claimed is:

1. A method for treating a condition in a patient which is treatable with noribogaine, noribogaine derivative, or pharmaceutically acceptable salt or solvate thereof while maintaining an acceptable QT interval prolongation in said patient, the method comprising:
a) administering to the patient an initial unit dose of noribogaine, noribogaine derivative, or pharmaceutically acceptable salt or solvate thereof, wherein said unit dose provides a therapeutic average serum concentration of 50 ng/mL to 180 ng/mL which serum concentration imparts an acceptable QT interval in said patient; and
b) maintaining said serum concentration by periodically administering at least one additional dose of noribogaine, noribogaine derivative, or pharmaceutically acceptable salt or solvate thereof, such that the at least one additional dose maintains the therapeutic average serum concentration of 50 ng/mL to 180 ng/mL during treatment;
wherein said additional dose or doses are continued as necessary to treat said condition, and further wherein said acceptable QT interval is no longer than 500 ms.

2. The method of claim 1, wherein the QT interval is prolonged by less than 50 ms.

3. The method of claim 1, wherein the QT interval is prolonged by less than 20 ms.

4. The method of claim 1, wherein the initial unit dose is higher than any of the at least one additional dose.

5. The method of claim 1, wherein the initial unit dose and at least one additional dose are incorporated into a single controlled release formulation.

6. The method of claim 1, wherein the initial unit dose is administered as subunit doses, which subunit doses are administered serially until the unit dose level is achieved, wherein the aggregate of subunit doses provides the initial unit dose and further provides the therapeutic average serum concentration.

7. The method of claim 1, wherein the initial unit dose is from 75 mg to 120 mg.

8. The method of claim 1, wherein the at least one additional dose is from 5 mg to 25 mg.

9. The method of claim 1, wherein the at least one additional dose is administered from 6 hours to 24 hours after the initial dose.

10. The method of claim 1, wherein at least two additional doses are administered, and further wherein the additional doses are administered from 6 hours to 24 hours after the previous dose.

11. The method of claim 1, further comprising selecting an addicted patient who is prescreened to evaluate tolerance for prolongation of QT interval.

12. A method for treating a condition in a patient which is treatable with noribogaine, noribogaine derivative, or pharmaceutically acceptable salt or solvate thereof while maintaining an acceptable QT interval prolongation in said patient, the method comprising:
a) administering to the patient an initial unit dose of noribogaine, noribogaine derivative, or pharmaceutically acceptable salt or solvate thereof, wherein said unit dose provides a therapeutic average serum concentration of 50 ng/mL to 180 ng/mL which serum concentration imparts an acceptable QT interval prolongation in said patient; and
b) maintaining said serum concentration by periodically administering at least one additional dose of noribogaine, noribogaine derivative, or pharmaceutically acceptable salt or solvate thereof, such that the at least one additional dose maintains the therapeutic average serum concentration of 50 ng/mL to 180 ng/mL during treatment;
wherein said additional dose or doses are continued as necessary to treat said condition, and further wherein said acceptable QT interval prolongation is no longer than 50 ms.

13. The method of claim 12, wherein the QT interval prolongation is less than 20 ms.

14. The method of claim 12, wherein the initial unit dose is higher than any of the at least one additional dose.

15. The method of claim 12, wherein the initial unit dose and at least one additional dose are incorporated into a single controlled release formulation.

16. The method of claim 12, wherein the initial unit dose is administered as subunit doses, which subunit doses are administered serially until the unit dose level is achieved, wherein the aggregate of subunit doses provides the initial unit dose and further provides the therapeutic average serum concentration.

17. The method of claim 12, wherein the initial unit dose is from 75 mg to 120 mg.

18. The method of claim 12, wherein the at least one additional dose is from 5 mg to 25 mg.

19. The method of claim 12, wherein the at least one additional dose is administered from 6 hours to 24 hours after the initial dose.

20. The method of claim 12, wherein at least two additional doses are administered, and further wherein the additional doses are administered from 6 hours to 24 hours after the previous dose.

21. The method of claim 12, further comprising selecting an addicted patient who is prescreened to evaluate tolerance for prolongation of QT interval.

22. A pharmaceutically acceptable controlled-release formulation comprising noribogaine, noribogaine derivative, or pharmaceutically acceptable salt or solvate thereof, such that the noribogaine is released over time, wherein the amount of noribogaine is sufficient to maintain a serum concentration of 50 ng/mL to 180 ng/mL when administered to a patient.

23. A kit of parts comprising two or more doses of noribogaine, noribogaine derivative, or pharmaceutically acceptable salt or solvate thereof, wherein the two or more doses comprise an amount of noribogaine, noribogaine derivative, or pharmaceutically acceptable salt or solvate thereof that is sufficient to maintain a serum concentration of 50 ng/mL to 180 ng/mL when administered to a patient.

24. The kit of parts of claim 23, wherein one dose comprises an initial dose of noribogaine, noribogaine derivative, or pharmaceutically acceptable salt or solvate thereof, said initial dose being sufficient to achieve a therapeutic serum concentration when administered to a patient; and at least one additional dose, said additional dose sufficient to maintain a therapeutic serum concentration when administered to a patient, wherein the therapeutic serum concentration is between 50 ng/mL and 180 ng/mL.

25. The kit of parts of claim 24, wherein the initial dose is from 75 mg to 120 mg.

26. The kit of parts of claim 24, wherein the at least one additional dose is from 5 mg to 25 mg.
